(19) United States
(12) Patent Application Publication
(10) Pub. No.: US 2003/0022253 A1
(43) Pub. Date: Jan. 30, 2003

(54) Method for identifying, isolating and producing neuroactive binding agents and binding agents derived thereby

(75) Inventor: Joseph R. Moskal, Evanston, IL (US)

Correspondence Address: Andrew W. Williams, McDonnell Boehnen Hulbert & Berghoff, 32nd Floor, 300 S. Wacker Drive, Chicago, IL, 60606, US

(73) Assignee: Nyxis Neurotherapies, Inc.

(21) Appl. No.: 10/205,236

(22) Filed: Jul. 25, 2002

Related U.S. Application Data
(60) Provisional application No. 60/307,740, filed on Jul. 25, 2001.

(51) Int. Cl. G01N 33/567; C12N 5/08

(52) U.S. Cl. 435/7.21; 435/368

(57) ABSTRACT

The present invention provides a method suitable for identifying and producing peptide and peptidomimetic molecules that are capable of producing a recognized phenotypic effect, even in cases where the endogenous peptide is not yet identified and compounds identified thereby. The invention further provides a method for generating related molecules that can be screened to identify a binding agent optimized for therapeutic use and compounds identified thereby. In a preferred embodiment, the invention provides a method for identifying and producing peptide and peptidomimetic molecules that are capable of modulating a phenotypic effect that is produced by the binding of another ligand to the same or a different receptor and compounds identified thereby.

### RELATED APPLICATION

This application claims the benefit of priority of U.S. Provisional Application No. 60/307,740 filed Jul. 25, 2001, which is incorporated by reference herein in its entirety.

### FIELD OF THE INVENTION

The present invention relates to methods for producing agents that bind to cell-surface receptors and the binding agents produced thereby.

### BACKGROUND OF THE INVENTION

Peptides and amino acids that bind to receptors on the surface of the cell membrane are used as agents to communicate signals between cells, most notably cells in the nervous system, endocrine system, and immune system. In some cases, the phenotypic effect of an exogenous ligand binding to the receptor may be recognized before the chemical structure of the endogenous binding agent is known. What is needed is a method suitable for identifying and producing amino acid, peptide, and peptidomimetic molecules that are capable of producing a recognized phenotypic effect, even in cases where the endogenous amino acid or peptide is not yet identified.

The central nervous system (CNS) of mammals uses many amino acids and peptides, the latter termed neuroactive peptides, for specialized signaling within the brain and spinal cord. Amino acid signaling molecules have been separated into two general classes: excitatory amino acids (glutamic acid, aspartic acid, cysteic acid and homocysteic acid), which depolarize neurons in the mammalian CNS, and inhibitory amino acids (gamma-aminobutyic acid, glycine, taurine and beta-alanine), which hyperpolarize mammalian neurons. (See generally, Cooper, J. R., et al., The Biochemical Basis of Neuropharmacology, 7th ed., Oxford University Press, New York, 1996, pp. 126-193). Among the better-known neuroactive peptides are vasopressin, oxytocin, somatostatin, corticotrophin releasing factor (CRF), growth hormone releasing hormone (GHRH), thyrotropin releasing hormone (TRH), cholecystokinin (CCK), vasoactive intestinal peptide (VIP), calcitonin gene related peptide (CGRP), glucagon, substance P and other tachykinins, opioid peptides (derivatives of proopiomelanocortin, proenkephalin and prodynorphin), neuropeptide Y (NPY), neurotensin, as well as cytokines. (See generally, Cooper, J. R., et al., 1996, pp.410-458).

The careful study of the complex signaling pathways that operate in the CNS, as well as diagnosis and therapy of CNS disorders, requires the identification and characterization of specific neuroactive peptides and their particular properties, as well as the characterization and localization of specific neurologically significant receptors. Identification of compounds that act as agonists and antagonists of CNS receptors, and characterization of such compounds as partial or complete, coordinately acting or independently acting, is thus useful. The ability to manipulate CNS receptor proteins increases with knowledge about specific neuroactive peptides and the behavior of CNS receptor complexes.

Most significantly, the identification of unique agonists or antagonists enables the fine characterization and localization of subsets of neuroactive receptors by the differential binding to these agonists or antagonists. By identifying neuroactive binding agents and using them to specifically perturb the behavior of known receptor complexes, a more detailed understanding becomes available about the receptor complex. In addition, the identification of new neuroactive binding agents offers alternative means of altering the behavior of known CNS receptor complexes, discovering previously unknown receptor complexes, or discovering unknown behavior of known receptors.

Recently, the World Health Organization predicted that within twenty years depression would become the second largest cause of disability and death worldwide. Already over one million people commit suicide each year out of a reported ten million attempts globally. Add to this people diagnosed with epilepsy, Alzheimer's disease, schizophrenia, and stoke, among others, and there are over 400 million people in the world today with mental and neurological disorders.

N-Methyl-D-asparate (NMDA) receptors are a subtype of excitatory amino acid receptors in mammalian brain, that gate an ion channel permeable to both Ca²⁺ and monovalent cations (MacDermott A. B., Mayer M. L., Westbrook G. L., Smith S. J., and Barker J. L. (1986) Nature 321, 519-522). This receptor is distinguished pharmacologically by a recognition site for the agonists glutamate and NMDA (Monaghan D. T. and Cotman C. W. (1986) Proc. Natl. Acad. Sci USA 83, 7532-7536) and the competitive antagonist D-(−)-2-amino-5-phosphonovaleric acid (D-AP5) (Olverman H. J., Jones A. W., and Watkins J. C. (1984) Nature 307, 4604-4662). The opening of the cation-selective channel, which is coupled to the agonist recognition site, can be regulated by glycine or by the voltage-dependent binding of magnesium (Johnson J. W. and Ascher P. (1987) Nature 325, 529-531). The binding of glycine to the NMDA receptor occurs at a pharmacologically unique site that is insensitive to strychnine and can be mimicked by serine or alanine (Reynolds I. J., Murphy S. N., and Miller R. J. (1987) Proc. Natl. Acad. Sci. USA 84, 7744-7748). Glycine increases the frequency of channel opening after binding and can regulate NMDA-receptor desensitization (Mayer M. L., Vyklicky L. Jr., and Clements J. (1989) Nature 338, 425-427). The anatomical colocalization of the binding of radiolabeled glycine and the NMDA receptor suggests a direct association of the glycine-binding site with the NMDA receptor-ion channel complex (Bristow D. R., Bowery N. G., and Woodruff G. N. (1986) Eur. J. Pharmacol. 126, 303-307). An additional binding site exists for psychoactive drugs such as phencyclidine (PCP) and the anticonvulsant MK-801 (Loo P. S., Brauwalder A. F. Lehmann J., Williams M., and Stills M. A. (1987) Mol. Pharmacol. 32, 820-830), which act noncompetitively to antagonize the action of NMDA (Anis N. A., Berry S. C., Burton N. R., and Lodge D. (1983) J. Pharmacol. 79, 565-575). The blocking action of these compounds is dependent on membrane potential and requires prior activation of the NMDA receptor (MacDonald J. F., Miljkovic Z., and Pennfeather P. (1987) J. Neurophysiol. 58, 251-265). This “use-dependency” is also apparent from measurement of radioligand binding at the PCP site (Loo P. S., Brauwalder A. F. Lehmann J., Williams M., and Stills M. A. (1987) Mol. Pharmacol. 32, 820-830; Bonhaus D. W. and McNamara J. O. (1988) Mol Pharmacol. 34, 250-255). Specific N-[1-(2-thienyl)cyclohexyl-3,4-[³H] piperidine([³H]TCP) binding can be potentiated by NMDA agonists (Loo P. S., Brauwalder A. F. Lehmann J., Williams M., and Stills M. A. (1987) Mol. Pharmacol. 32, 820-830). These data suggest that the accessibility of PCP to its binding site is increased when the NMDA receptor is in the open conformation (Bonhaus D. W. and McNamara J. O. (1988) Mol Pharmacol. 34, 250-255), consistent with a PCP-binding site within the channel.

The N-methyl-D-aspartate (NMDA) receptor appears to be an excellent target for the development of cognitive enhancers. It acts as a modulator of synaptic transmission that can trigger synapses to function more efficiently (Collingridge, G. L. and Singer, W. (1990) TIPS 11(7), 290-296). For example, activation of the NMDA receptor is critical for the induction of long-term potentiation (LTP), the formation of spatial memories, and associative learning (Morris, R. G. M. (1989) J. Neurosci. 9, 3040-3057). A loss of NMDA-receptor function, on the other hand, occurs in normal aging brain and has been implicated in the learning deficits that often occur during aging and Alzheimer's disease (Proctor, A. W., Wong, E. H. F., Stratmann, G. C., Lowe, S. L. and Bowen, D. M. (1989) J. Neurochem. 53, 698-704).

Unlike virtually all other ligand-gated ion channels, the NMDA receptor-ion channel complex is unique in requiring the occupation of two distinct recognition sites by glutamate and glycine for activation. Recent data suggests that glycine acts as a modulator of glutamate synaptic transmission. This makes the glycine site a potentially important target for the development of cognitive enhancing therapeutics, as well as anticonvulsants and neuroprotective agents (Kemp, J. A. and Leeson, P. D. (1993) TIPS 14, 20-25).

Accordingly, there has been a surge in the development of pharmacological agents that interact selectively with the glycine-binding site. The partial agonist, D-cycloserine, has been shown to have cognitive-enhancing properties in vivo (Hood, W. F., Compton, R. P. and Monahan, J. B. (1989). Neurosci. Letters. 98, 91-95 Monahan J. B., Corpus V. M., Hood W. F., Thomas J. W., and Compton R. P. (1989) J. Neurochem 53, 370-375), as has the glycine prodrug, Milacemide (Handelmann, G. E., Nevins, M. E., Muellers, L. L., Arnolde, S. M. and Cordi, A. A. (1989) Pharm. Biochem. Behav. 34, 823-828). Both of these compounds, however, appear to result in desensitization with chronic administration (Quartermain D., Mower, J., Rafferty, M. F., Herting, R. L. and Lanthorn, T. H. (1994) Euro. J. Pharm. 257(1-2), 7-12). These reports collectively suggest several general observations. First, it is feasible to create agonists acting at the glycine site of the NMDA receptor. Second, such agonists can have a significant effect on cognitive enhancement in ways that suggest clinical relevance. Finally, there is clearly a need for new classes of agonists with greater in vivo efficacy.

### SUMMARY OF THE INVENTION

The present invention provides methods for isolating phenotypically active binding agents and methods for identifying binding agent mimics.

In a first aspect, the invention provides a method for identifying a phenotypically active binding agent, the method comprising:

(a) contacting a first cell with a first binding agent, wherein said first cell comprises a binding target;

(b) detecting binding of said first binding agent to said first cell;

(c) preparing a second binding agent, wherein said second binding agent is derived from said first binding agent;

(d) contacting a second cell with said second binding agent; wherein said second cell comprises said binding target;

(e) detecting a phenotypic change in said second cell in response to said contact with said second binding agent; wherein said phenotypic change in said second cell is in response to said contact with said second binding agent.

In a preferred embodiment, the first cell and the second cell are neural cells. In a more preferred embodiment, the second binding agent is a protein, a peptide, or a peptidomimetic. In certain embodiments, the first binding agent is an antibody molecule or fragment thereof, preferably a monoclonal antibody; and more preferably, the active region is at least a fragment of a complementarity determining region of the antibody. In other embodiments, the first binding agent is a fragment of an antibody expressed on a phage particle. In other embodiments, the phenotypic change comprises the induction of long-term potentiation. In certain embodiments, the second binding agent comprises a sequence of contiguous amino acids that is substantially identical to a region of contiguous amino acids in the first binding agent, and preferably the second binding agent comprises a region of at least 3, at least 4, at least 5, or at least 9 contiguous amino acids. In other embodiments, the second binding agent comprises a sequence of contiguous amino acids that is substantially identical to a retro-inverso peptide corresponding to a contiguous sequence of amino acids in the first binding agent. In certain embodiments, the method further comprises the step of isolating a nucleic acid encoding the active region of the first binding agent, preferably where the first binding agent is expressed by a hybridoma cell, and more preferably where the nucleic acid is a fragment of either genomic DNA or a cDNA prepared form the mRNA of the hybridoma cell.

In a second embodiment, the invention provides a method for identifying a phenotypically active binding agent, the method comprising:

(a) identifying a binding target;

(b) contacting a first cell with a first binding agent, wherein said first cell comprises said binding target;

(c) detecting binding of said first binding agent to said first cell;

(d) preparing a second binding agent, wherein said second binding agent is derived from said first binding agent;

(e) contacting a second cell with said second binding agent; wherein said second cell comprises said binding target;

(f) detecting a phenotypic change in said second cell in response to said contact with said second binding agent; wherein said phenotypic change in said second cell is in response to said contact with said second binding agent.

In a third embodiment, the invention provides a method for identifying a phenotypically active binding agent, the method comprising:

(a) identifying a first binding agent, wherein first binding agent binds to a binding target,

(b) preparing a second binding agent, wherein said second binding agent is derived from said first binding agent;

(c) contacting a cell with said second binding agent; wherein said cell comprises said binding target;

(d) detecting a phenotypic change in said cell in response to said contact with said second binding agent; wherein said phenotypic change in said second cell is in response to said contact with said second binding agent.

In a preferred embodiment, the cell is a neural cells. In a more preferred embodiment, the second binding agent is a protein, a peptide, or a peptidomimetic. In certain embodiments, the first binding agent is an antibody molecule or fragment thereof, preferably a monoclonal antibody; and more preferably, the active region is at least a fragment of a complementarity determining region of the antibody. In other embodiments, the first binding agent is a fragment of an antibody expressed on a phage particle. In other embodiments, the phenotypic change comprises the induction of long-term potentiation. In certain embodiments, the second binding agent comprises a sequence of contiguous amino acids that is substantially identical to a region of contiguous amino acids in the first binding agent, and preferably the second binding agent comprises a region of at least 3, at least 4, at least 5, or at least 9 contiguous amino acids. In other embodiments, the second binding agent comprises a sequence of contiguous amino acids that is substantially identical to a retro-inverso peptide corresponding to a contiguous sequence of amino acids in the first binding agent. In certain embodiments, the method further comprises the step of isolating a nucleic acid encoding the active region of the first binding agent, preferably where the first binding agent is expressed by a hybridoma cell, and more preferably where the nucleic acid is a fragment of either genomic DNA or a cDNA prepared form the mRNA of the hybridoma cell.

In a fouth embodiment, the invention provides a method for identifying a phenotypically active binding agent, the method comprising:

(a) contacting a first cell with a first binding agent, wherein said first cell comprises a binding target and wherein said first binding agent is encoded by a nucleic acid;

(b) detecting binding of said first binding agent to said first cell;

(c) preparing a nucleic acid library, wherein said nucleic acid library comprises said nucleic acid encoding said first binding agent, or a fragment thereof;

(d) preparing a second binding agent, wherein said second binding agent is prepared by utilizing said nucleic acid library;

(e) contacting a second cell with said second binding agent; wherein said second cell comprises said binding target;

(f) detecting a phenotypic change in said second cell in response to said contact with said second binding agent; wherein said phenotypic change in said second cell is in response to said contact with said second binding agent.

In a preferred embodiment, the second binding agent is neuroactive. Preferably, the first cell and the second cell are neural cells. More preferably, the nucleic acid library is derived from the genomic DNA or from the cDNA of a hybridoma that expresses a monoclonal antibody. In certain embodiments, the nucleic acid library is a phage display library or a stochastically generated library. More preferably, the nucleic acid library is modified by gene shuffling, or the binding agent mimic is prepared by gene shuffling. In preferred embodiments, the binding agent is an antibody, preferably a monoclonal antibody. In certain embodiments, the second binding agent is a peptide, preferably substantially identical to at least a fragment of complementarity determining region of an antibody. In other embodiments, the second binding agent is a peptidomimetics, preferably a retro-inverso peptide having substantially identical activity as at least a fragment of the complementarity-determining region of an antibody. In other embodiments, the second binding agent is an organic molecule having substantially identical activity as at least a fragment of the complementarity-determining region of an antibody.

### BRIEF DESCRIPTION OF THE DRAWINGS

FIG. 1. Monoclonal antibody G6E3 identifies specific neurons in the hippocampus and cerebellum in the adult rat. Coronal section through the hippocampal formation of an adult rat brain. Intensely stained cells are evident in both the stratum pyramidale (not shown) and the stratum granulosum (X360).

FIG. 2. Monoclonal antibody G6E3 stains embryonic hippocampal neurons in tissue sections and in culture. a, Unfixed, frozen, 10 μm section of 19-20 d embryonic rat brain stained sequentially with G6E3 and a fluoresceinated secondary antibody. Staining is apparent in the pyramidal cell layer (p), as well as in the area of the developing dentate gyrus (g) (X150). b, Phase-contrast photomicrograph of a 2.5-week-old rat hippocampal culture. Neuron of various sizes and morphologies are evident on a confluent background on non-neuronal cell types (X200). c, Three-week-old hippocampal culture stained with G6E3 followed by a fluoresceinated secondary antibody (X360). A typical field of G6E3-positive cells was photographed, and the cultures were than processed for NSE reactivity. Visualization of specific staining was effected by the PAP methods of Stemberger ((1974) Immunocytochemistry, Prentice-Hall, Englewood Cliffs, N.J.). d, Same field as in c showing that all G6E3-positive cells stain for NSE (X360). e, Three-week-old hippocampal culture stained with G6E3 as in c. G6E3-positive cells are shown. The culture was then fixed and stained with anti-GAD antibodies followed by a rhodaminated secondary antibody (X360). f, Same field as in c showing the G6E3-positive cells are surrounded by GAD-positive terminals (X360).

FIG. 3. Experimental arrangement for application of antibody B6B21 to field CA1 pyramidal cell apical dendrites. Two recording microelectrodes (2 M NaCl) were positioned 200-400 μm apart in the CA 1 pyramidal cell layer. A bipolar stimulating electrode was placed in the stratum radiatum. One recording site was then randomly selected for the B6B21-containing pipette, which was placed in the apical dendrites in the stratum radiatum 400-500 μm away from and at the same depth as the recording electrode. The other recording site served as the control evoked response.

FIG. 4. B6B21 application to CA1 pyramidal cell apical dendrites enhanced LTP production. A: typical evoked population responses recorded from paired sites in CA1 pyramidal cell body layer (stratum pyramidale). B6B21 was applied to the apical dendritic field of one of the two sites (B6B21 sites), whereas the other (200-500 μm apart) served as the paired control. Responses are shown before (light traces) and 30 min after (dark traces) high-frequency afferent stimulation (10 bursts of 100 Hz×5 pulses each). Traces are superimposed to illustrate the significantly greater LTP of the population spike response observed at the site that received B6B21 ejection (4×500 msec) 15 min before tetanization, relative to the untreated site. Calibration bar=5 mV/2 msec; filled circles denotes stimulus artifacts. B: Time course in another experiment, showing population spike amplitude at control (open circles) and B6B21-treated (filled circles) recording sites in the CA1 cell body layer. B6B21 was applied 15 min before high-frequency Schaffer collateral stimulation. After stimulation, LTP at the antibody-treated site was significantly greater than the control, untreated site. C: Mean±SEM LTP (% of prestimulated baseline) in field CA1 at control (solid bar, n=8 slices) and B6B21-treated sites (hatched bar; n=8 slices). B6B21 applied 15 min before high-frequency stimulation significantly enhanced the magnitude of LTP induced by submaximal stimulation (*p<0.01, paired t test).

FIG. 5. Stimulation of [³H]TCP binding by B6B21. Measurements of specific [³H]TCP binding (10 nM) were performed under non-equilibrium conditions (1 h, 25° C.). B6B21 (25 μg/ml) was purified from hybridoma-conditioned media using protein A-Sepharose. Mouse IgG (25 μg/ml) was a commercial preparation. Experiments were performed in the absence or in the presence of a mixture of glutamate, glycine, and magnesium acetate (50 μM of each). Control values (100%) correspond to specific [³H]TCP binding measured in the absence of any NMDA ligands or mAb. Error bars represent standard errors of the mean. p<0.05 vs. control, paired t test. There was no significant change in [³H]TCP binding elevation induced by NMDA ligand alone or in combination with mAbs.

FIG. 6. Modulation of [³H]TCP binding by NMDA agonists. Samples were incubated in the presence of 3 nM [³H]TCP for 1 h at 25° C. Dose-response curves were generated using the following: (A) glutamate (filled circles), glutamate plus 50 μM glycine (open circles), or glutamate plus 25 μg/ml of B6B21 (open squares), and (B) NMDA (filled triangles) or NMDA plus 25 μg/ml of B6B21 (open triangles). Data are expressed as a percentage of increase in [³H]TCP binding (% of maximum binding in each of the assay conditions used). The data are of a representative experiment that was replicated three times. The maximum binding under various conditions used was (picomoles per milligram of protein) as follows: control, 63; glutamate, 132; glutamate+glycine, 310; glutamate+B6B211, 286; NMDA, 130; NMDA+B6B21, 305.

FIG. 7. Inhibition of [³H]TCP binding by NMDA and glycine antagonists. A: Dose-dependent inhibition of [³H]TCP binding by 7-CL KYNA. Experiments were performed in the absence (filled circles) or in the presence of 60 μg/ml of B6B21 (open circles), with no addition of glutamate and glycine. B: Dose-dependent inhibition of [³H]TCP binding by D-AP5. Washed membranes were incubated with 5 nM [³H]TCP for 1 hr at 25° C. and various concentrations of D-AP5 in the absence (filled circles), or in the presence of 50 μM glutamate (open circles), 50 μM glycine (filled triangles), or 40 μg/ml of B6B21 (open squares). Results are expressed as a percentage of specific [³H]TCP binding observed in the absence of antagonists. Data shown are from a representative experiment that was repeated twice.

FIG. 8. Inhibition of [³H]glycine binding by B6B21. [³H]Glycine binding was measured by (A) competition of 50 nM [³H]glycine with various concentrations of unlabeled glycine or (B) direct radioligand binding using 10-1,000 nM [³H]glycine. The binding experiments were performed in the absence and in the presence of 0.12 mg/ml of B6B21 or 0.24 mg/ml of B6B21. Nonspecific binding was determined in the presence of 1 mM unlabeled glycine. Membranes (100-150 μg of protein) were incubated at 25° C. for 1 hr, and the reaction were terminated by the centrifugation method previously described. Linear regression analysis of the Scatchard plots (B) indicated a best fit to a single population of sites (r=0.9). Data are expressed as a percentage of specific [³H]glycine bound in the absence of competing cold glycine. Note that B6B21 increased the apparent K_{D} for [³H]glycine at this site by 188% at a concentration of 240 μg/ml, whereas B6B21 stimulated [³H]TCP binding twofold at a concentration of 25 μg/ml.

FIG. 9. Trace eye-blinking condition was facilitated by daily intraventricular infusions of the monoclonal antibody B6B21. a, mean learning curves (±S.E.M.) and least-squares fitted lines for each curve are shown, expressed as the percentage of conditioned responses per block of 40 training trials. Using the linear interpolation algorithms of Igor (WaveMetrics, Lake Oswego, Oreg.), each curve was normalized to the mean number of trials required to reach the criterion (80% CR) level for that group, so that the qualitative summaries of learning rates for animals that required different numbers of trials to reach criterion could be made. For statistical purpose, the slopes of the individual non-normalized learning curves were co-paired, indicating that B6B21 treated rabbits learned significantly faster (P<0.003) than rabbits in either control group (whose learning curves overlapped). b, Comparisons to a number of trials required to reach the criterion level, number of trials to reach a simpler criterion of 8/10 conditioned responses, and number of trials to gain two consecutive blocks of 8/10 conditioned responses all indicated significant facilitation of learning in the trace conditioning task by B6B21 (P<0.01)*.

METHODS. Monoclonal antibodies were made against freshly dissected 5 day postnatal rat dentate gyri and purified using Protein A-Sepharose chromatography¹³⁵. Eluates were immediately neutralized, dialysed, and concentrated. Concentrated antibody (typically 1-2 ml) was again dialysed against two changes (each 2 l) of HEPES buffer to remove endogenous glycine contamination. The antibodies were sterilized by filtration through a 0.22-μm filter and frozen at −80° C. in small aliquots. All antibody injections were given under sterile conditions. Neither SDS-polyacrylamide gel electrophoresis nor low molecular mass high-performance liquid chromatography (HPLC) of purified antibodies revealed any glycine-like contaminants. Specifically, no glycine or other α-amino acid contaminants were detected either in fresh samples of antibody or in aliquots of the solution infused intraventricularly, even when samples were subjected to several freeze-thaw cycles (minimal HPLC detection threshold of 0.1 pmol per injection). Commercial IgG antibodies were used which had no glycine-like activity, and gave similar negative results from HPLC. Cannulated rabbits were always paired with an animal from one of the other two groups, with six animals used in each group. For 5 min immediately before each day's training, rabbits simultaneously received 5 μl infusion in each ventricle of either B6B21 (1 μg μl ⁻¹) suspended in artificial cerebral spinal fluid (aCSF; composition, in mM; 124 NaCl; 26 NaHCO₃; 3KCl; 2.4 CaCl₂; 1.3 MgSo₄; 1.24NaH₂O₄; 10 D-glucose (pH 7.4)), of aCSF alone, or of mouse IgG (1 μg μl⁻¹ Sigma) in aCSF at a rate of 1 μl ventricle⁻¹ min⁻¹. The treatment received by the subjects was unknown to the trainer. Trace nictitating membrane conditioning (using a 100 ms duration, 6 kHz, 85 db binaural tone condition stimulus (CS) followed, after a 500 ms interstimulus trace interval by a 150 msec corneal air-puff unconditioned stimulus (US)) began immediately after infusion with 80 trials per day.

FIG. 10. Peptide sequences synthesized based on the amino acid sequence of the hypervariable regions of the monoclonal antibody B6B21. NT1-3 are the hypervariable sequences of the B6B21 light chain. NT-3 was found to mimic the monoclonal antibody. NT-4-18 are peptides derived from this sequence. The conditions used to assess peptidomimetic activity are as follows:

The [³H]MK-801 binding assay is a function assay in that increased [³H]MK801 binding can only occur upon receptor-induced channel opening, since the binding domain of Mk-801 is inside the ionophore of the NMDA receptor complex. We have further modified the assay by adding 7-clorokynurenic acid, a selective antagonist to the glycine binding site on the NMDA receptor-channel complex, to our reaction mixtures. The ability of NT-3 to reverse the inhibition of [³H]MK-801 binding strongly suggests that the peptide is acting at the glycine binding site.

Membrane preparation. Crude synaptic membranes were prepared using rat hippocampal tissue (male Sprague-Dawley rats) and extensively washed using procedures described previously. Briefly, tissue had been stored at −80° C. was homogenized in ice cold 5 mM Tris (pH 7.4) using a Brinkman Polytron and then pelleted by centrifugation at 48,000 g for 20 min. The resulting supernatant was discarded and the membranes were washed an additional 3×. pellets were then resuspended in 5 mM EDTA/15 mM Tris (pH 7.4) and incubated for 1 hr at 37° C. The membrane suspensions were then pelleted by centrifugation at 48,000 g for 20 min and stored at −80° C. until the day of the assay.

Receptor binding assay. Frozen pellets were thawed at room temperature and washed 3× by resuspension in 5 mM Tris (pH 7.4) and centrifugation. Final pellets were suspended at concentrations of 2-3 mg/ml in 5 mM Tris buffer (pH 7.4). Binding reactions were initiated by the addition of 200 kg of freshly prepared membranes to reaction mixtures (1 ml final volume) containing 1 nM [³H]MK-801 at 25° C. in the presence of a range of peptide concentrations and 60 l M 7-chlorokynurenic acid. Non-specific binding in the presence of 10 μM unlabeled MK-801. Binding reaction were terminated by filtration through a Brandel 24-well cell harvester onto Whatman GF/B glass filters that had been presoaked in 0.25% polyethyleneimine for 30 min.

FIG. 11. The structure of NT-13 is threonine-proline-proline-threonine. Using Insight II 95.0 (Molecular Simulations, San Diego, Calif.) software, NT-13 appears to exist in a beta-type conformation when in solution, as shown.

FIG. 12. Monoclonal antibody-derived custom peptides (MADCP). This cartoon depicts the general method that can be used to generate therapeutically useful compounds via monoclonal antibodies. By identifying appropriate targets as immunogens coupled with the proper screening methods, antibodies can be generated and the hypervariable regions (CDRs) can be cloned and sequenced. From this information, small peptides that possess therapeutic value can easily be synthesized. Moreover, thousands of variants can be readily synthesized via amino acid substitutions and because these peptides tend to be quite small, non-peptide mimetics can also be synthesized.

### DETAILED DESCRIPTION OF THE INVENTION

The present invention provides methods for identifying phenotypically active binding agents. For the purposes of the invention, “phenotypically active binding agents” include, but are not limited to, antibodies (monoclonal and polyclonal), peptides (such as neuroactive peptides, proteins, peptidomimetics, and other non-peptide organic molecules, and phage particles. The methods of the present invention can be used to identify new families of compounds that have clinical potential with respect to various biological disorders, such as, but not limited to, neurological disorders.

Unique binding agents capable of inducing a phenotypic change in a cell are identified through the methods of this invention. A method of the present invention allows a first binding agent, such as a peptide or monoclonal antibody, to contact a cell that contains a binding target, such as a cell-surface receptor. It should be understood that a binding agent of the invention is meant to encompass both individual binding agents, such as a specific monoclonal antibody, and a plurality of binding agents, such as a panel of antibodies or a library of peptides. After the binding of the agent to the cell is detected, a second binding agent is prepared, such as a peptide, peptidomimetic, or phage particle, with the knowledge of the first binding. Thus, the second binding agent is derived from the first binding agent, for example, but not limited to, through the cloning of the complementarity determining region of a monoclonal antibody. Again, the second binding agent of the invention is meant to include either a specific binding agent, such as a synthesized peptide, or a plurality of binding agents, such as a panel of peptides or a library of peptide-expressing phage particles. The second binding agent, or agents, are screened to identify agents that can cause a phenotypic change in a cell that comprises a binding target. The phenotypic change can be detected by different methods, including, but not limited to, assaying for formation of long-term potentiation.

As an example of the present invention, in an attempt to create a new family of compounds that would have clinical potential with respect to neurological disorders, a method or the invention was developed for creating and screening monoclonal antibodies for their ability to affect learning and memory. This method identified B6B21, a “functional” antibody able to affect learning and memory, which specifically modulates one member of the glutamate family of receptors, the N-methyl-D-aspartate receptor. This monoclonal antibody was subsequently used as a platform to create a family of small peptide mimetics. Other methods of this invention can be used, among other things, to create additional “functional” antibodies and to create additional families of small peptide and peptidomimetics. Within this application, unless otherwise stated, the techniques employed in the inventive method can be found in any of several well-known references including: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). All references cited are herein incorporated by reference in their entirety.

For the purposes of this invention, a “binding agent” is defined as a molecule, or plurality of molecules, capable of binding to, among other things, a plasma membrane receptor molecule on a cell. Binding agents include, but are not limited to, proteins, peptides, antibodies, phage particles, and peptidomimetics. Binding agents of the present invention are phenotypically active, meaning that when the agent binds to a cell, for example a peptide-responsive cell, the result is a phenotypic change in the cell. The binding agents of the present invention can contain an active region, which is a region of the binding agent that is responsible for the activity or function of the binding agent. The active region can include the entire binding agent, a segment of the binding agent, or a plurality of segments of the binding agent. For example, if the binding agent is a protein, the active region can be the entire protein, a segment of the protein, or a plurality of segments, which may or may not be contiguous in the protein's polypeptide chain. As example of an active region is the complementarity determining region of a monoclonal antibody, or a fragement thereof. One skilled in the art would understand that what constitutes a binding agent, or an active region of a binding agent, is not limited to the examples provided herein.

The binding agent of the present invention is not limited to a single binding agent, but instead encompasses both a single binding agent and a plurality of binding agent. Therefore, the use of the singular form of the word is meant to encompass the plural. For example a binding agent includes, but is not limited, a single monoclonal antibody, a panel of monoclonal antibodies, a single peptide, and a panel of peptides, a single peptide-expression phage, a library of phage that express a plurality of peptides, a single peptidomimetic, and a panel of peptidomimetics.

For the purposes of this invention, a cell is intended to include, but is not necessarily limited to, cells bearing a plasma membrane receptor molecule that can bind an agent, such as a peptide, wherein binding of the agent to the receptor results in a phenotypic change in the cell. Cells of the present invention include neural cell, and more specifically cells of the brain. A neural cell includes a cell that pertains to a nerve or nerves, or to the brain or central nervous system. Those skilled in the art will recognize that a cell of the present invention includes cells that contain exogenous nucleic acid (e.g., DNA or RNA), from which a protein or proteins are expressed, even when the protein or proteins are not normally expressed in the cell. For example, Xenopus oocytes can be made to express plasma membrane receptor molecules or functional ionic channels after injection of mRNA (see, for example, Leonard and Kelso, 1990, Neuron, 2:53-60), and therefore are a cell or the present invention.

For the purposes of this invention, a “binding target” is intended to encompass any biological molecule that is capable of binding to a phenotypically active binding agent. Preferably, the binding target of located on, in, or near a cellular membrane. More preferably, the binding target is located on or near the cell surface. More prefereably, the binding target is a cell-surface receptor. In addition, the binding target could be, or could be associated to, an ion channel, such as a voltage-gated ion channel. Preferably, the binding molecule of the present invention is a the N-methly-D-aspartate receptor. For the purposes of this invention, it is not necessary that the binding target be identified or isolated.

The methods of the present invention utilize a phenotypically active binding agent that is capable of binding to a binding target. For the purposes of this invention, “binding target” is intended to include cell surface receptors.

The binding of the first binding agent to the first cell is detected by conventional means. For example, the binding can be detected direct means, such as be labeling the first binding agent with, for example, a radiolabel or a chemical label. Alternatively, the detection can be indirect, for example, by using immunological methods, such as, but not limited to, flow cytometric analysis (FACS), immunoblot analysis, and radioimmunoassay. The immunological reagents for use in the immunological methods can be detected directly or indirectly, using fluorescence, chemical reactions, or radiolabeling. Alternatively, the binding of the first binding agent can be detected by measuring a phenotypic change in the first cell, wherein this phenotypic change can be the same, substantially the same, or different than the phenotypic change detected in the second cell in response to binding of the second binding agent.

For the purposes of the invention, phenotypic changes in the second cell in response to the second binding agent include, but are not limited to, changes in membrane conductance, membrane current, membrane potential, exocytosis, endocytosis, cellular motility, contractility, gene expression, protein expression, protein secretion, intracellular signaling activity, kinase activity, intracellular phosphorylation, association of membrane proteins, and cytoskeleton rearrangement. Phenotypic changes can be assayed directly or indirectly. One skilled in the art will recognize methods by which any of the above phenotypic changes can be assayed. For example, phenotypic change resulting from binding to the NMDA receptor can be detected by measuring the induction of LTP, measuring TCP binding, and measuring glycine binding.

The second binding agent of the invention is derived from the first binding agent, or, in other words, a physical characteristic of the first binding agent is used prepare the second binding agent, or agents. For example, if the first binding agent is a protein, peptide, or antibody, the primary amino acid sequence can be used to derive the second binding agents. Preferably, the second binding agent is a protein, polypeptide, or peptidomimetic. The primary amino acid sequence or sequences of the second binding agent can be identical to the first binding agent, or fragment thereof. Preferably, the second binding agent comprises a peptide that is a sequence of contiguous amino acids that is substantially identical to a region of contiguous amino acids in the first binding agent. More preferably, the second binding agent is a peptide that comprises a sequence that is at least 3, 4, 5, 6, 7, 8, or 9 contiguous amino acids identical to a region of contiguous amino acids in the first binding agent. In addition, the second binding agent can be a peptide that is identical to the first binding agent, or fragment thereof, or it can contain amino acid substitutions, insertions, additions and/or deletions, wherein the substations may be conservative or non-conservative, or any combination thereof. For example, a “conservative amino acid substitution” involves a substitution of a native amino acid residue with a non-native residue such that there is little or no effect on the polarity or charge of the amino acid residue at that position. Furthermore, any native residue in the polypeptide may also be substituted with alanine, as has been previously described for “alanine scanning mutagenesis.” Conservative amino acid substitutions also encompass non-naturally occurring amino acid residues that are typically incorporated by chemical peptide synthesis rather than by synthesis in biological systems. These include peptidomimetics and other reversed or inverted forms of amino acid moieties.

Naturally occurring residues may be divided into classes based on common side chain properties:

1) hydrophobic: norleucine, Met, Ala, Val, Leu, Ile;

2) neutral hydrophilic: Cys, Ser, Thr;

3) acidic: Asp, Glu;

4) basic: Asn, Gln, His, Lys, Arg;

5) residues that influence chain orientation: Gly, Pro; and

6) aromatic: Trp, Tyr, Phe.

For example, non-conservative substitutions may involve the exchange of a member of one of these classes for a member from another class. Such substituted residues may be introduced into one or more regions of a polypeptide.

In making such changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al., 1982, J. Mol. Biol. 157:105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0 +1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred. One may also identify epitopes from primary amino acid sequences on the basis of hydrophilicity. These regions are also referred to as “epitopic core regions.”

Desired amino acid substitutions (whether conservative or non-conservative) can be determined by those skilled in the art at the time such substitutions are desired. Alternatively, derivation of the first binding agent can utilize the nucleic acid encoding the binding agent, or a fragment thereof, if it is so encoded, including DNA, genomic DNA, cDNA, RNA, and mRNA. The nucleic acid encoding the first binding agent can be sequenced, to determine the primary structure of both the nucleic acid and the expressed polypeptide. This nucleic acid or polypeptide sequence can then be used to derive the second binding agent. Alternatively, processes utilizing a variety of methods such as sequential random mutagenesis, error-prone mutagenesis or gene shuffling can also be utilized to derive the second binding agent. For example, a binding agent encoded by a nucleic acid can be subjected to directed evolution, which does not necessarily require any prior knowledge of the structure-function relationship. An exemplary directed evolution procedure includes: selection of the gene, creation of the variant library, insertion of the library into an expression vector, expression of the gene library to produce binding agent libraries, screening of the binding agents for the property of interest, and isolation of the nucleic acid corresponding to the improved variant properties so that the cycle can be repeated as desired. Thus, the generation and screening of binding agents with improved performance is preferably carried out in iterative steps. After several cycles, the performance of modified binding agents should be optimized. One exemplary system is provided by WO98/51802, incorporated herein by reference in its entirety. Many suitable systems for performing such development cycles are available to one of skill in the art. Certain non-limiting examples of such systems are reviewed below.

One such method, disclosed in U.S. Pat. No. 5,605,793 (incorporated herein by reference in its entirety) describes the random fragmentation of a template DNA sequence and re-assembly in the presence of a partially random oligonucleotide having overlapping sequence with the template. In this manner, novel libraries of DNA sequences are produced.

Another method for recombining sequences is described in U.S. Pat. Nos. 5,811,238; 5,830,721, and 5,837,458 (each of which is being incorporated herein by reference in their entirety) wherein a DNA sequence is randomly fragmented, “shuffled” to randomly recombine the sequences, and screened to identify DNA sequences encoding proteins or peptides having particular characteristics. This cycle is then repeated to further select for enzymes having even more evolved characteristics.

Yet another method for generating large numbers of randomly associated DNA sequences encoding enzymes is provided by U.S. Pat. No. 5,965,408 (incorporated herein by reference in its entirety). As shown therein, “sexual” PCR is performed using a template DNA and random oligonucleotide primers and interrupting synthesis of the template DNA molecule. In this way, novel DNA sequences are provided that encode enzymes having unique characteristics.

It is also possible to generate functional polypeptides by randomly assembling small oligonucleotides, such as described by U.S. Pat. No. 5,723,323; U.S. Pat. No. 5,814,476; U.S. Pat. No. 5,817,483; U.S. Pat. No. 5,824,514; and U.S. Pat. No. 5,976,862, each of which being hereby incorporated by reference in their entirety. As shown therein, polypeptides can be generated using randomly generated 7-mer and/or 8-mer oligonucleotides, for example, to generate larger “random” DNA sequences. These sequences can then be cloned into expression vectors, which are then transformed into the appropriate host cell. The host cells are then screened for expression of binding agents and the DNA encoding the binding agents isolated. Following isolation of such DNA molecules, the binding agent can be studied further, and potentially further manipulated using the techniques described herein.

Alternatively, the first binding agent can be used to derive the second binding agent, wherein the second binding agent is a peptidomimetic compound, that is, a wholly or partially non-peptide compound having the essential three-dimensional shape and chemical reactivity. The invention also provides methods for selecting such peptidomimetic compounds.

A peptidomimetic is a molecule that mimics the biological activity of a peptide but is no longer peptidic in chemical nature. Peptidomimetic compounds are known in the art and are described, for example, in U.S. Pat. No. 6,245,886. By strict definition, a peptidomimetic is a molecule that no longer contains any peptide bonds (that is, bonds between amino acids). However, the term “peptidomimetic” as used herein is broadly defined to encompass molecules that are no longer completely peptidic in nature. Examples of molecules falling under this broader definition, wherein part of a peptide molecule is replaced by a structure lacking peptide bonds, are described below. Whether completely or partially non-peptide, peptidomimetics broadly defined according to this invention provide a spatial arrangement of reactive chemical moieties that closely resembles the three-dimensional arrangement of active groups in the peptide on which the peptidomimetic is based. As a result of this similar active-site geometry, the peptidomimetic has effects on biological systems that are similar to the biological activity of the peptide.

The peptidomimetics of the present invention are preferably substantially similar in both three-dimensional shape and biological activity to the peptides set forth above. Substantial similarity means that the geometric relationship of groups in the peptide that react with the corresponding receptor is preserved and at the same time, that the peptidomimetic can stimulate the phenotypic change elicited by the corresponding peptide binding agent.

Preferably, the efficacy of the peptidomimetic molecule in stimulating the phenotypic change is at least about 0.1 fold, more preferably at least about 0.2 fold, optimally at least about 0.5 fold of the efficacy of at least one of the peptide binding agents of this invention.

There are clear advantages for using a peptidomimetic of a given peptide rather than the peptide itself, because peptides commonly exhibit two undesirable properties: (1) poor bioavailability; and (2) short duration of action. Peptidomimetics offer an effective route around these two major obstacles, since the molecules concerned are small enough to be both orally active and have a long duration of action. There are also considerable cost savings and improved patient compliance associated with peptidomimetics, since they can be administered orally compared with parenteral administration for peptides. Furthermore, peptidomimetics are much cheaper to produce than peptides. Finally, there are problems associated with stability, storage and immunoreactivity for peptides that are not experienced with peptidomimetics.

Thus peptides described above have utility in the development of such small chemical compounds with similar biological activities and therefore with similar therapeutic utilities. The techniques of developing peptidomimetics are conventional. Thus, peptide bonds can be replaced by non-peptide bonds that allow the peptidomimetic to adopt a similar structure, and therefore biological activity, to the original peptide. Further modifications can also be made by replacing chemical groups of the amino acids with other chemical groups of similar structure. The development of peptidomimetics can be aided by determining the tertiary structure of the original peptide, either free or bound to the corresponding receptor, by NMR spectroscopy, crystallography and/or computer-aided molecular modeling. These techniques aid in the development of novel compositions of higher potency and/or greater bioavailability and/or greater stability than the original peptide [Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference]. Once a potential peptidomimetic compound is identified, it may be synthesized and assayed using the phenotypic change of the peptide-responsive cell to assess its activity.

Thus, through use of the methods described above, the present invention provides second binding agents exhibiting enhanced therapeutic activity in comparison to the first binding agents. The peptidomimetic compounds obtained by the above methods, having the biological activity of the above named peptides and similar three-dimensional structure, are encompassed by this invention. It will be readily apparent to one skilled in the art that a peptidomimetic can be generated from any of the modified peptides described above or from a peptide bearing more than one of the modifications described above. It will furthermore be apparent that the peptidomimetics of this invention can be further used for the development of even more potent non-peptidic compounds, in addition to their utility as therapeutic compounds.

Specific examples of peptidomimetics derived from the peptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect peptide structure and biological activity. The reduced isostere pseudopeptide bond is a suitable pseudopeptide bond that is known to enhance stability to enzymatic cleavage with no or little loss of biological activity (Couder, et al. (1993), Int. J. Peptide Protein Res., 41:181-184, incorporated herein by reference). Thus, the amino acid sequences of these peptides may be identical to the sequences of the L-amino acid peptides identified or produced by the method of the present invention, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above).

To confer resistance to proteolysis, peptide bonds may also be substituted by retroinverso pseudopeptide bonds (Dalpozzo, et al. (1993), Int. J. Peptide Protein Res., 41:561-566, incorporated herein by reference). According to this modification, the amino acid sequences of the peptides may be identical to the sequences of the L-amino acid peptides described above, except that one or more of the peptide bonds are replaced by a retroinverso pseudopeptide bond. Preferably the most N-terminal peptide bond is substituted, since such a substitution will confer resistance to proteolysis by exopeptidases acting on the N-terminus. The synthesis of peptides with one or more reduced retro-inverso pseudopeptide bonds is known in the art (Dalpozzo, et al. (1993), cited above).

One example of such a second binding agent or binding agent mimic is a “retro-inverso”, “retro-inverso isomer”, or “retro-inverso isomer of a binding agent”. As used herein, the term “D-amino acid isomers” refer to binding agents in which the one or more of the L-isomer amino acids have been replaced with the corresponding D-isomer amino acid. As used herein, the term “retro-inverso”, “retro-inverso isomer”, or “retro-inverso isomer of a binding agent”, is intended to encompass binding agents in which the sequence of the amino acids is reversed as compared to the sequence in the corresponding natural binding agent, and all L-amino acids are replaced with D-amino acids. For example, if a parent peptide is Tyr-Ile-His, the retro-inverso form is D-His-D-Ile-D-Tyr. Compared to the parent peptide, a retro-inverso peptide has a reversed backbone while retaining substantially the original spatial conformation of the side chains, resulting in a retro-inverso isomer with a topology that closely resembles the parent peptide. For further description of retro-inverso peptides, see Goodman et al. “Perspectives in Peptide Chemistry” pp. 283-294 (1981) and Sisto, U.S. Pat. No. 4,522,752, each of which being herein incorporated by reference in their entirety.

The present invention also provides for active agents that correspond in sequence to the aforementioned binding agents of the present invention, wherein one or more of the L-amino acids have been substituted by the corresponding D-amino acids (“D-amino acid isomers”). In addition, one can employ retro-inverso isomers or partially modified retroinverso isomers of the binding agents of the invention. As defined earlier, retro-inverso isomers are isomers in which the direction of the sequence is reversed and the chirality (i.e. “handedness”) of each amino acid residue is inverted. Partially modified retroinverso isomers are isomers in which only some of the peptide bonds are reversed and the chirality of the amino acid residues in the reversed portion is inverted. Retro-inverso isomers and partially modified retro-inverso isomers are described further by Chorev, M. and Goodman, M., TIBTECH, Vol. 13:438-444, 1995, herein incorporated by reference in its entirety. The retro-inverso derivatives can be prepared by the procedure of Briand et al., J. Biol. Chem., 270:20686-20691, 1995, herein incorporated by reference in its entirety.

Peptoid derivatives of peptides represent another form of modified peptides that retain the important structural determinants for biological activity, yet eliminate the peptide bonds, thereby conferring resistance to proteolysis (Simon, et al., 1992, Proc. Natl. Acad. Sci. US., 89:9367-9371 and incorporated herein by reference). Peptoids are oligomers of N-substituted glycines. A number of N-alkyl groups have been described, each corresponding to the side chain of a natural amino acid. The peptidomimetics of the present invention include compounds in which at least one amino acid, a few amino acids or all amino acid residues are replaced by the corresponding N-substituted glycines.

Further peptidomimetics are described in U.S. Pat. No. 6,251,864. Such peptidomimetics include peptides having one or more of the following modifications:

1 peptides wherein one or more of the peptidyl [—C(O)NR—] linkages (bonds) have been replaced by a non-peptidyl linkage such as

a. a—CH₂-carbamate linkage [—CH₂—OC(O)NR—];

b. a phosphonate linkage;

c. a—CH₂-sulfonamide [—CH₂—S(O)₂ NR—] linkage;

d. a urea [—NHC(O)NH—] linkage;

e. a—CH₂-secondary amine linkage; or

f. an alkylated peptidyl linkage [—C(O)NR⁶— where R⁶ is lower alkyl];

2. peptides wherein the N-terminus is derivatized to

a. a —NRR¹ group;

b. to a—NRC(O)R group;

c. to a—NRC(O)OR group;

d. to a—NRS(O)₂ R group;

e. to a—NHC(O)NHR group where R and R¹ are hydrogen or lower alkyl with the proviso that R and R¹ are not both hydrogen;

f. to a succinimide group;

g. to a benzyloxycarbonyl-NH—(CBZ—NH—) group; or

h. to a benzyloxycarbonyl-NH— group having from 1 to 3 substituents on the phenyl ring selected from the group consisting of lower alkyl, lower alkoxy, chloro, and bromo; or

3. peptides wherein the C terminus is derivatized to —C(O)R² where R² is selected from the group consisting of hydroxy, lower alkoxy, and —N R³R⁴ where R³ and R⁴ are independently selected from the group consisting of hydrogen and lower alkyl.

Accordingly, preferred peptides and peptide mimetics comprise a compound having:

a molecular weight of less than about 5000 daltons, and

effective in producing the corresponding phenotypic change elicited by the corresponding peptide binding agent,

wherein from zero to all of the —C(O)NH— linkages of the peptide have been replaced by a linkage selected from the group consisting of a—CH₂OC(O)NR— linkage; a phosphonate linkage; a—CH₂S(O)₂ NR— linkage; a—CH₂NR— linkage; and a—C(O)N NR⁶— linkage; and a—NHC(O)NH— linkage where R is hydrogen or lower alkyl and R⁶ is lower alkyl,

further wherein the N-terminus of said peptide or peptide mimetic is selected from the group consisting of a—NRR¹ group; a—NRC(O)R group; a—NRC(O)OR group; a —NRS(O)₂R group; a—NHC(O)NHR group; a succinimide group; a benzyloxycarbonyl-NH—group; and a benzyloxycarbonyl-NH— group having from 1 to 3 substituents on the phenyl ring selected from the group consisting of lower alkyl, lower alkoxy, chloro, and bromo, where R and R¹ are independently selected from the group consisting of hydrogen and lower alkyl,

and still further wherein the C-terminus of said peptide or peptide mimetic has the to formula —C(O)R² where R² is selected from the group consisting of hydroxy, lower alkoxy, and —N R³R⁴ where R³ and R⁴ are independently selected from the group consisting of hydrogen and lower alkyl and where the nitrogen atom of the —N R³R⁴ group can optionally be the amine group of the N-terminus of the peptide so as to form a cyclic peptide,

and physiologically acceptable salts thereof.

In a related embodiment, the invention is directed to a labeled peptide or peptidomimetic comprising a peptide or peptidomimetic described as above having covalently attached thereto a label capable of detection.

The binding agents of the present invention are preferably administered to a patient as pharmaceutically acceptable compositions. The binding agents may be administered orally (including bucally and sublingually), parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrastemal, infusion techniques or intraperitoneally. Suppositories for rectal administration of the binding agent may also be prepared by mixing the it with a suitable non-irritating excipient such as cocoa butter and polyethylene glycols that are solid at ordinary temperatures but liquid at the rectal temperature and will therefore melt in the rectum and release the drug.

The dosage regimen for treating a patient with the binding agents of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The pharmaceutically acceptable compositions of this invention can be processed in accordance with conventional methods of pharmacy to produce medicinal agents for administration to patients, including humans and other mammals. For oral administration, the pharmaceutical composition may be in the form of, for example, a capsule, a tablet, a suspension, or liquid. The pharmaceutical composition is preferably made in the form of a dosage unit containing a given amount of binding agent. A suitable daily dose for a human or other mammal may vary widely depending on the condition of the patient and other factors, but, once again, can be determined using routine methods. The vector may also be administered by injection as a composition with suitable carriers including saline, dextrose, or water.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A suitable topical dose of active ingredient of a binding agent of the present invention is administered one to four, preferably two or three times daily. For topical administration, the binding agent may comprise from 0.001% to 10% w/w, e.g., from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes) and drops suitable for administration to the eye, ear, or nose.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc. Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

While the binding agents of the invention can be administered as the sole active pharmaceutical agent, they can also be used in combination with one or more binding agents of the invention or other agents. When administered as a combination, the therapeutic agents can be formulated as separate compositions that are given at the same time or different times, or the therapeutic agents can be given as a single composition.

For the purposes of this application, a transcriptional regulatory region is defined as any region of a gene involved in regulating transcription of a gene, including, but not limited to promoters, enhancers and repressors. A transcriptional regulatory element is defined as any element involved in regulating transcription of a gene, including but not limited to promoters, enhancers and repressors. A promoter is a regulatory sequence of DNA that is involved in the binding of RNA polymerase to initiate transcription of a gene. A gene is a segment of DNA involved in producing a peptide, polypeptide or protein, including the coding region, non-coding regions preceding (“leader”) and following (“trailer”) the coding region, as well as intervening non-coding sequences (“introns”) between individual coding segments (“exons”). Coding refers to the representation by the nucleic acid of amino acids, start and stop signals in a three base “triplet” code. Promoters are often upstream (“5′ to”) the transcription initiation site of the corresponding gene. Other regulatory sequences of DNA in addition to promoters are known, including sequences involved with the binding of transcription factors, including response elements that are the DNA sequences bound by inducible factors. Enhancers comprise yet another group of regulatory sequences of DNA that can increase the utilization of promoters, and can function in either orientation (5′-3′ or 3′-5′) and in any location (upstream or downstream) relative to the promoter. Preferably, the regulatory sequence has a positive activity, i.e., binding of an endogenous ligand (e.g. a transcription factor) to the regulatory sequence increases transcription, thereby resulting in increased expression of the corresponding target gene. The term operably linked refers to the combination of a first nucleic acid fragment representing a transcriptional control region having activity in a cell joined to a second nucleic acid fragment encoding a reporter or effector gene such that expression of said reporter or effector gene is influenced by the presence of said transcriptional control region.

A responsive element is a portion of a transcriptional control region that induces expression of a nucleotide sequence following the interaction of a cell with a compound. There may be multiple responsive elements within a single transcriptional control region and each of these elements may function independently of any other elements of that transcriptional control region. Thus, a responsive element may be incorporated into a reporter gene vector independent from the remainder of the transcriptional control region from which it is derived and function to drive expression of the reporter gene under the proper conditions.

The terms overexpressed or underexpressed typically relate to expression of a nucleic acid sequence or protein at a higher or lower level, respectively, than that level typically observed in a cell (i.e., normal control). In certain cases, the terms overexpressed or underexpressed may also relate to the expression level in a cell that has been contacted by a compound as compared to the expression level in a similar cell that has not been contacted by the compound.

For the purposes of this application, hybridization is typically performed under stringent conditions. The term “stringent conditions” refers to hybridization and washing under conditions that permit only binding of a nucleic acid molecule such as an oligonucleotide or cDNA molecule probe to highly homologous sequences. For example, a stringent wash solution is 0.015 M NaCl, 0.005 M NaCitrate, and 0.1% SDS used at a temperature of 55° C.-65° C. Another stringent wash solution is 0.2×SSC and 0.1% SDS used at a temperature of between 50° C.-65° C.

A DNA or amino acid sequence is identical to another sequence where the sequences are identical. A DNA or amino acid sequence is substantially identical or substantially the same as another sequence where the sequences are 50-100% identical. In a preferred embodiment, substantially identical sequences share 60-100% identity, more preferably 70-100% identity, even more preferably 80-100% identity and even more preferably 90-100% identity. In a most preferred embodiment, substantially identical sequences share 95-100% identity.

The term antibody in its various grammatical forms is used herein to refer to immunoglobulin molecules and immunologically active portions of immunoglobulin molecules, i.e., molecules that contain an antibody-combining site or paratope. Exemplary antibody molecules are intact immunoglobulin molecules, substantially intact immunoglobulin molecules and portions of an immunoglobulin molecule, including those portions known in the art as Fab, Fab′, F(ab′)₂ and F(v). The complementarity determining regions (CDR) of an antibody comprise the parts of the molecule that determine their specificity and make contact with a specific ligand.

For the purposes of this invention, the term immunological reagent is intended to encompass antisera and antibodies, including monoclonal antibodies, as well as fragments therefore (F(ab), Fab′, F(ab′)₂, and F(v) fragments). Also include in the definition of immunological reagent are chimeric antibodies, humanized antibodies, and recombinantly produced antibodies and fragments thereof, as well as aptamers (i.e., oligonucleotides capable of interacting with target molecules such as peptides). Immunological methods used in conjunction with the methods of the invention include direct and indirect (for example, sandwich-type) labeling techniques, immunoaffinity columns, immunomagnetic beads, fluorescence activated cell sorting (FACS), enzymelinked immunosorbant assays (ELISA), and radioimmune assay (RIA). The immunological reagents can be labeled using fluorescence, antigenic, radioisotopic or biotin labels, among others, or a labeled secondary or tertiary immunological detecting reagent can be used to detect binding of the immunological reagent.

A polypeptide refers to an amino acid sequence encoded by a nucleic acid, or a fragment thereof.

The word inoculum in its various grammatical forms is used herein to describe a composition containing a polypeptide of this invention as an active ingredient used for the preparation of antibodies against the polypeptide. When a polypeptide is used in an inoculum to induce antibodies it is to be understood that the polypeptide can be used in various embodiments, e.g., alone or linked to a carrier as a conjugate, or as a polypeptide polymer. However, for ease of expression and in context of a polypeptide inoculum, the various embodiments of the polypeptides of this invention are collectively referred to herein by the term polypeptide and its various grammatical forms.

An antibody of the present invention is typically produced by immunizing a mammal with an inoculum containing a polypeptide and thereby induce in the mammal antibody molecules having immunospecificity for immunizing polypeptide. The antibody molecules are then collected from the mammal and isolated to the extent desired by well known techniques such as, for example, by using DEAE Sephadex or Protein G to obtain the IgG fraction.

Exemplary antibody molecules for use with the present invention are intact immunoglobulin molecules, substantially intact immunoglobulin molecules and those portions of an immunoglobulin molecule that contain the paratope, including those portions known in the art as Fab, Fab′, F(ab′)₂ and F(v). Fab and F(ab′)₂ portions of antibodies are prepared by the proteolytic reaction of papain and pepsin, respectively, on substantially intact antibodies by methods that are well known. See for example, U.S. Pat. No. 4,342,566 to Theofilopolous and Dixon. Fab′ antibody portions are also well known and are produced from F(ab′)₂ portions followed by reduction of the disulfide bonds linking the two heavy reduction of the disulfide bonds linking the two heavy chain portions as with mercaptoethanol, and followed by alkylation of the resulting protein mercaptan with a reagent such as iodoacetamide.

The preparation of antibodies against polypeptide is well known in the art. See Staudt et al., J. Exp. Med., 157:687-704 (1983), or the teachings of Sutcliffe, J. G., as described in U.S. Pat. No. 4,900,811, the teaching of which are hereby incorporated by reference. Briefly, to produce a peptide antibody composition of this invention, a laboratory mammal is inoculated with an immunologically effective amount of a polypeptide of this invention typically as present in a vaccine of the present invention. The anti-polypeptide antibody molecules thereby induced are then collected from the mammal and those immunospecific for both a polypeptide and the corresponding recombinant protein are isolated to the extent desired by well known techniques such as, for example, by immunoaffinity chromatography.

To enhance the specificity of the antibody, the antibodies are preferably purified by immunoaffinity chromatography using solid phase-affixed immunizing polypeptide. The antibody is contacted with the solid phase-affixed immunizing polypeptide for a period of time sufficient for the polypeptide to immunoreact with the antibody molecules to form a solid phase-affixed immunocomplex. The bound antibodies are separated from the complex by standard techniques.

For a polypeptide that contains fewer than about 35 amino acid residues, it is preferable to use the peptide bound to a carrier for the purpose of inducing the production of antibodies. One or more additional amino acid residues can be added to the amino- or carboxy-termini of the polypeptide to assist in binding the polypeptide to a carrier. Cysteine residues added at the amino- or carboxy-termini of the polypeptide have been found to be particularly useful for forming conjugates via disulfide bonds. However, other methods well known in the art for preparing conjugates can also be used. The techniques of polypeptide conjugation or coupling through activated functional groups presently known in the art are particularly applicable. See, for example, Aurameas, et al., Scand. J. Immunol., Vol. 8, Suppl. 7:7-23 (1978) and U.S. Pat. No. 4,493,795, No. 3,791,932 and No. 3,839,153. In addition, a site-directed coupling reaction can be carried out so that any loss of activity due to polypeptide orientation after coupling can be minimized. See, for example, Rodwell et al., Biotech., 3:889-894 (1985), and U.S. Pat. No. 4,671,958. Exemplary additional linking procedures include the use of Michael addition reaction products, di-aldehydes such as glutaraldehyde, Klipstein, et al., J. Infect. Dis., 147:318-326 (1983) and the like, or the use of carbodiimide technology as in the use of a water-soluble carbodiimide to form amide links to the carrier. Alternatively, the heterobifunctional cross-linker SPDP (N-succinimidyl-3-(2-pyridyldithio) proprionate)) can be used to conjugate peptides, in which a carboxy-terminal cysteine has been introduced.

Useful carriers are well known in the art, and are generally proteins themselves. Exemplary of such carriers are keyhole limpet hemocyanin (KLH), edestin, thyroglobulin, albumins such as bovine serum albumin (BSA) or human serum albumin (HSA), red blood cells such as sheep erythrocytes (SRBC), tetanus toxoid, cholera toxoid as well as polyamino acids such as poly D-lysine:D-glutamic acid, and the like. The choice of carrier is more dependent upon the ultimate use of the inoculum and is based upon criteria not particularly involved in the present invention. For example, a carrier that does not generate an untoward reaction in the particular animal to be inoculated should be selected.

The present inoculum contains an effective, immunogenic amount of a polypeptide of this invention, typically as a conjugate linked to a carrier. The effective amount of polypeptide per unit dose sufficient to induce an immune response to the immunizing polypeptide depends, among other things, on the species of animal inoculated, the body weight of the animal and the chosen inoculation regimen is well known in the art. Inocula typically contain polypeptide concentrations of about 10 micrograms (μg) to about 500 milligrams (mg) per inoculation (dose), preferably about 50 micrograms to about 50 milligrams per dose. The term “unit dose” as it pertains to the inocula refers to physically discrete units suitable as unitary dosages for animals, each unit containing a predetermined quantity of active material calculated to produce the desired immunogenic effect in association with the required diluent; i.e., carrier, or vehicle. The specifications for the novel unit dose of an inoculum of this invention are dictated by and are directly dependent on (a) the unique characteristics of the active material and the particular immunologic effect to be achieved, and (b) the limitations inherent in the art of compounding such active material for immunologic use in animals, as disclosed in detail herein, these being features of the present invention.

Inocula are typically prepared from the dried solid polypeptide-conjugate by dispersing the polypeptide-conjugate in a physiologically tolerable (acceptable) diluent such as water, saline or phosphate-buffered saline to form an aqueous composition. Inocula can also include an adjuvant as part of the diluent. Adjuvants such as complete Freund's adjuvant (CFA), incomplete Freund's adjuvant (IFA) and alum are materials well known in the art, and are available commercially from several sources.

The antibody so produced can be used, inter alia, to detect a polypeptide in a sample such as a tissue section or body fluid sample. Anti-polypeptide antibodies that inhibit function of the polypeptide can also be used in vivo in therapeutic methods as described herein. A preferred anti-polypeptide antibody is a monoclonal antibody. The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g., a bispecific monoclonal antibody.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler and Milstein, Nature, 256:495-497 (1975), the description of which is incorporated by reference. The hybridoma supernates so prepared can be screened for the presence of antibody molecules that immunoreact with a polypeptide.

Briefly, to form the hybridoma from which the monoclonal antibody composition is produced, a myeloma or other self-perpetuating cell line is fused with lymphocytes obtained from the spleen of a mammal hyperimmunized with a antigen, such as is present in a polypeptide of this invention. The polypeptide-induced hybridoma technology is described by Niman et al., Proc. Natl. Acad. Sci., USA, 80:4949-4953 (1983), the description of which is incorporated herein by reference. It is preferred that the myeloma cell line used to prepare a hybridoma be from the same species as the lymphocytes. Typically, a mouse of the strain 129 GIX⁺ is the preferred mammal. Suitable mouse myelomas for use in the present invention include the hypoxanthine-aminopterinthymidine-sensitive (HAT) cell lines P3X63-Ag8.653, and Sp2/O-Ag14 that are available from the American Type Culture Collection, Rockville, Md., under the designations CRL 1580 and CRL 1581, respectively. Splenocytes are typically fused with myeloma cells using polyethylene glycol (PEG) 1500. Fused hybrids are selected by their sensitivity to HAT. Hybridomas producing a monoclonal antibody of this invention are identified using the enzyme linked immunosorbent assay (ELISA) described in the Examples.

A monoclonal antibody of the present invention can also be produced by initiating a monoclonal hybridoma culture comprising a nutrient medium containing a hybridoma that produces and secretes antibody molecules of the appropriate polypeptide specificity. The culture is maintained under conditions and for a time period sufficient for the hybridoma to secrete the antibody molecules into the medium. The antibody-containing medium is then collected. The antibody molecules can then be further isolated by well known techniques. Media useful for the preparation of these compositions are both well known in the art and commercially available and include synthetic culture media, inbred mice and the like. An exemplary synthetic medium is Dulbecco's Minimal Essential Medium (DMEM; Dulbecco et al., Virol. 8:396 (1959)) supplemented with 4.5 gm/1 glucose, 20 mM glutamine, and 20% fetal calf serum. An exemplary inbred mouse strain is the Balb/c. Other methods of producing a monoclonal antibody, a hybridoma cell, or a hybridoma cell culture are also well known. See, for example, the method of isolating monoclonal antibodies from an immunological repertoire as described by Sastry, et al., Proc. Natl. Acad. Sci. USA, 86:5728-5732 (1989); and Huse et al., Science, 246:1275-1281 (1989).

The monoclonal antibodies of this invention can be used in the same manner as disclosed herein for antibodies of the present invention. For example, the monoclonal antibody can be used in the therapeutic, diagnostic or in vitro methods disclosed herein. Also contemplated by this invention is the hybridoma cell, and cultures containing a hybridoma cell that produce a monoclonal antibody of this invention.

It is also possible to isolated antibodies reactive against polypeptides of the instant invention using phage display techniques. Display of antibody fragments on the surface of viruses which infect bacteria (bacteriophage or phage) makes it possible to produce human sFvs with a wide range of affinities and kinetic characteristics. To display antibody fragments on the surface of phage (phage display), an antibody fragment gene is inserted into the gene encoding a phage surface protein (pIII) and the antibody fragment-pII fusion protein is expressed on the phage surface (McCafferty et al. (1990) Nature, 348: 552-554; Hoogenboom et al. (1991) Nucleic Acids Res., 19: 4133-4137). For example, a sFv gene coding for the V.sub.H and V.sub.L domains of an anti-lysozyme antibody (D1.3) was inserted into the phage gene III resulting in the production of phage with the DI.3 sFv joined to the N-terminus of pIII thereby producing a “fusion” phage capable of binding lysozyme (McCafferty et al (1990) Nature, 348: 552-554). The skilled artisan may also refer to Clackson et al. (1991) Nature, 352: 624-628), (Marks et al. (1992) Bio/Technology, 10: 779-783), Marks et al Bio/Technology, 10: 779-785 (1992) for further guidance. In the instant case, the antibody fragment gene is isolated from the immunized mammal, and inserted into the phage display system. Phage containing antibodies reactive to the polypeptide are then isolated and characterized using well-known techniques. Kits and services are available for generating antibodies by phage display from well-known sources such as Cambridge Antibody Technology Group plc (United Kingdom).

Autoantibodies to the polypeptides of the instant invention may also be detected using techniques well-known and widely available to the skilled artisan. For detection of autoantibodies in the serum of a patient by an antigen-antibody reaction, various conventional immunologically methods can be used such as a method of directly measuring a reaction in a liquid phase and a solid phase and a method of measuring an inhibitory reaction immunologically by adding an inhibiting substance. The following are the examples of the above-mentioned detecting methods, (1) aggregation reaction; (2) DID: double immune diffusion method (Octarony method); (3) ELISA: enzyme linked immunoabsorbent assay, (4) FIA: fluorescent immunosorbent assay, (5) nephlometry method, (6) radioimmuno assay (RIA), (7) immunofluorescent methods. Such methods are described in available references such as U.S. Pat. No. 5,976,810.

For the purposes of this invention, the term “immunological reagents” is intended to encompass antisera and antibodies, particularly monoclonal antibodies, as well as fragments thereof (including F(ab), F(ab)₂, F(ab)N and Fᵥ fragments). Also included in the definition of immunological reagent are chimeric antibodies, humanized antibodies, and recombinantly-produced antibodies and fragments thereof, as well as aptamers (i.e., oligonucleotides capable of interacting with target molecules such as peptides). Immunological methods used in conjunction with the reagents of the invention include direct and indirect (e.g, sandwich-type) labeling techniques, immunoaffinity columns, immunomagnetic beads, fluorescence activated cell sorting (FACS), enzyme-linked immunosorbent assays (ELISA), and radioimmune assay (RIA), most preferably FACS. For use in these assays, the detectable immunological reagents can be labeled, using fluorescence, antigenic, radioisotopic or biotin labels, among others, or a labeled secondary or tertiary immunological detection reagent can be used to detect binding of the detectable immunological reagents (i.e., in secondary antibody (sandwich) assays).

While a preferred form of the invention has been shown in the drawings and described, since variations in the preferred form will be apparent to those skilled in the art, the invention should not be construed as limited to the specific form shown and described, but instead is as set forth in the claims.

In an effort to develop new drugs for the treatment of neurological disorders, the following antibody technology was developed. Firstly, it was assumed that complex neural functions such as learning and memory emerge from and are controlled by more fundamental cellular and molecular processes. Secondly, it is well established that monoclonal antibodies (MAbs) have the capacity to bind with very high specificity to their targets or antigenic determinants. Thus, it seemed possible that by selecting the appropriate target and screening strategy MAbs could be generated that bound to specific targets, modulated characterizable molecular processes, and perturbed complex cognitive functions.

The described methodology of this technology, termed Monoclonal Antibody Direct Custom Peptide Synthesis, is summarized as follows. Fresh unfrozen membrane preparations of micropunched, five-day postnatal, rat dentate gyri of the developing hippocampus were used as the target for generating the panel of MAbs. Screening the panel proceeded as follows: (1) Only IgG's were used; (2) MAbs were then chosen that bound to unfixed frozen sections of adult rat hippocampus; (3) The resultant sub-panel were screened for MAbs that bound to the cell surfaces of unfixed primary hippocampal cultures; (4) Those surviving the previous screen were then evaluated for their ability to modulate long-term potentiation (LTP) using hippocampal slice preparations; (5) LTP-modulators were then assessed for their ability to modulate N-methyl-D-aspartate receptor function and finally (6) one MAb, B6B21, was evaluated for its effects on learning and memory, in vivo, using a rabbit hippocampus-dependent trace eyeblink conditioning paradigm; (7) the complimentary determining regions (CDRs) of B6B21 was cloned and the synthetic peptide NT-13 was produced from this cloned region; (8) NT-13 was shown to improve learning and memory and prevent neural degeneration during stroke in animal models.

### Example 1

### Production and Screening of Monoclonal Antibodies

Monoclonal antibodies specific to the developing rat dentate gyrus were produced by the following methods. Five-day post-natal rat dentate gyri were removed by the micropunch method of Palkovits and Brownstein (Palkovits M. and Brownstein M. J. (1983) in Methods in Neuroscience, Microdissection Techniques (Cuello A. C. ed.), Wiley, Chichester, UK. Pp 1-36). The tissue was immediately homogenized in PBS at 4° C. and injected intraperitoneally into Balb/c mice. An average of 50 mg wet weight of tissue was injected per mouse. This procedure was repeated 4 times over 2 months. Monoclonal antibodies were generated by conventional techniques using NS-1 as the parent myeloma cell line (Kohler G. and Milstein C. (1976) Immunol. 6, 511-519). The antibodies were screened against unfixed frozen tissue that had been cryoprotected by preincubation with a graded series (10-30%) of sucrose in Dulbecco's modified Eagle's medium (high glucose; Gibco, Grand Island N.Y.). Ten micron sections (cut with a cryostat and placed on collagen-coated glass microscope slides) and were incubated for 24 hr at 4° C. with G6E3 hybridoma conditioned tissue culture medium (RPMI plus 15% fetal bovine serum), tissue culture medium alone as a control, or medium containing the nonspecific IgG₁ monoclonal antibody P3X63-Ag8 as a second control. Antibody binding was visualized by incubating sections with a fluoresceinated rabbit anti-mouse IgG (Dako Corp., Santa Barbara, Calif.) diluted 1:50 with PBS. Antibodies were further screened with 50 μm vibratome sections from rat brains that had been perfused with a solution of ice-cold 0.2% picric acid and 4% paraformaldehyde in 0.167 M solution phosphate buffer, pH 7.0. In these experiments, the secondary antibody was a peroxidase-conjugate goat anti-mouse IgG (Boehringer-Mannheim, Indianapolis, Ind.) diluted 1:100 in PBS and visualized with diaminobenzidine following the method described by Kliss et al. ((1984) Proc. Natl. Acad. Sci USA 81, 1854-1858). Sub-typing of the antibodies was done using a Subtype Immunoglobulin Kit from Boehringer-Mannheim.

To identify MAbs which bind specifically to the hippocampus, a secondary screen was performed with hippocampal cultures. Hippocampi were removed from 19-20 d rat embryos, mechanically dissociated, and plated at a density of 0.5-1.0×10⁶ cells per 35 mm collagen-coated dish. Plating medium consisted of minimal essential medium with Eagle's salts (MEM), glucose (final concentration, 30 mM), sodium bicarbonate (final concentration, 38 mM) (Advanced Biotechnologies, Rockville, Md.), 5% fetal bovine serum, 5% horse serum (both from Hazelton Dutchland, Inc., Denver, Pa.), and serum-free additives. The serum-free additives were prepared by a modification of Romijn et al. ((1984) Neurosci. Behav. Rev. 8, 301-334) and were composed of glutamine (final concentration 2 mM), insulin (5 μg/ml) (Collaborative Research, Lexington, Mass.), corticosterone (100 ng/ml), sodium selenite (3×10⁻⁸ M), transferrin 50 μm/ml), and thyroxine (5 ng/ml) (all from Sigma, St. Louis, Mo.). After 4-6 d, when dividing cells had grown to confluency, the medium was changed to MEM, serum free additives, 5% horse serum, fluorodeoxyuridine (FUDR; final concentration, 10⁻⁵ M) and uridine (U; final concentration, 10⁻⁴ M) (both from Sigma) to inhibit further growth of dividing cells. The medium was replaced after 3-4 d and twice weekly thereafter with MEM, serum-free additives, and horse serum without FUDR or U. Cultures were maintained in a humidified atmosphere of 92% air: 8% carbon dioxide at 36.5° C.

2-to 3-week old, unfixed, hippocampal cultures were stained with G6E3 conditioned medium (CM) or ascites fluid for 45 min to 1 hr on ice. Ascites fluid was diluted 1:10 to 1:50 in Hank's balanced salt solution (Gibco) with 10 mM HEPES, and 1% bovine serum albumin (HBSA). Conditioned medium was used full strength or diluted 1:1 in HBSA. After rinsing in HBSA, cultures were incubated for 30 minutes on ice with a fluoresceinated secondary antibody [affinity-purified F(ab′)₂ goat anti-mouse IgG; Jackson ImmunoResearch, Avondale, Pa] diluted 1:50 or 1:100 in HBSA. It was necessary to stain unfixed cultures on ice to preserve antigenicity and prevent patching and capping of antigen-antibody complexes. After rinsing in HBSA, cultures were viewed immediately in Zeiss Photomicroscope III (Baltimore Instruments, Baltimore, Md.). For double-labeling experiments, cultures were fixed after G6E3 staining with 4% paraformaldehyde in 0.1 M phosphate buffer, pH 7.3, at 4° C. for 1 hr. Cultures were then incubated with anti-NSE (Marangos P. J., Zomzely-Neurath C., and York C. (1975) Arch. Biochem. Biophys. 170, 289-293; a gift from Dr. Paul Marangos) antibodies followed by an unlabeled sheep anti-rabbit secondary antibody. Specific staining was visualized by the peroxidase-antiperoxidase (PAP) method of Stemberger (Stemberger L. A. (1974) Immunocytochemistry, Prentice-Hall, Englewood Cliffs, N.J.). Cultures were also incubated with anti-GAD antibodies (Oertel W. H., Schmechel D. E., Mugnaini E., Tappaz M. L., and Kopin I. J. (1981) Neuroscience 6, 2715-2735; a gift from Dr. Wolfgang Oertel) followed by a rhodaminated rabbit anti-sheep secondary antibody (from Jackson ImmunoResearch as above). For double staining with G6E3 and anti-NSE it was necessary to photograph G6E3-positive cells after fixation, because the diaminobenzidine reaction product occluded the fluorescent staining. The same fields were rephotographed after PAP staining.

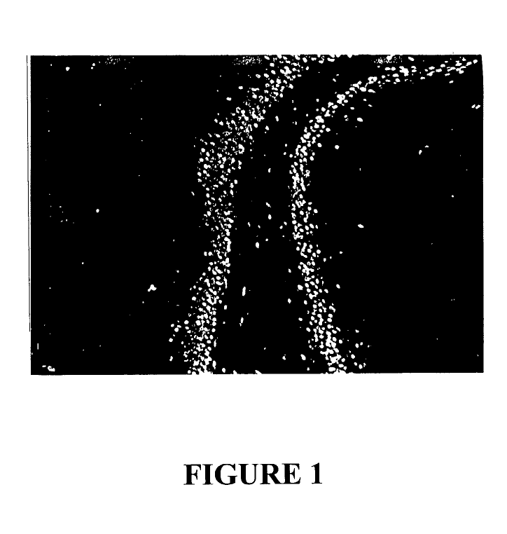

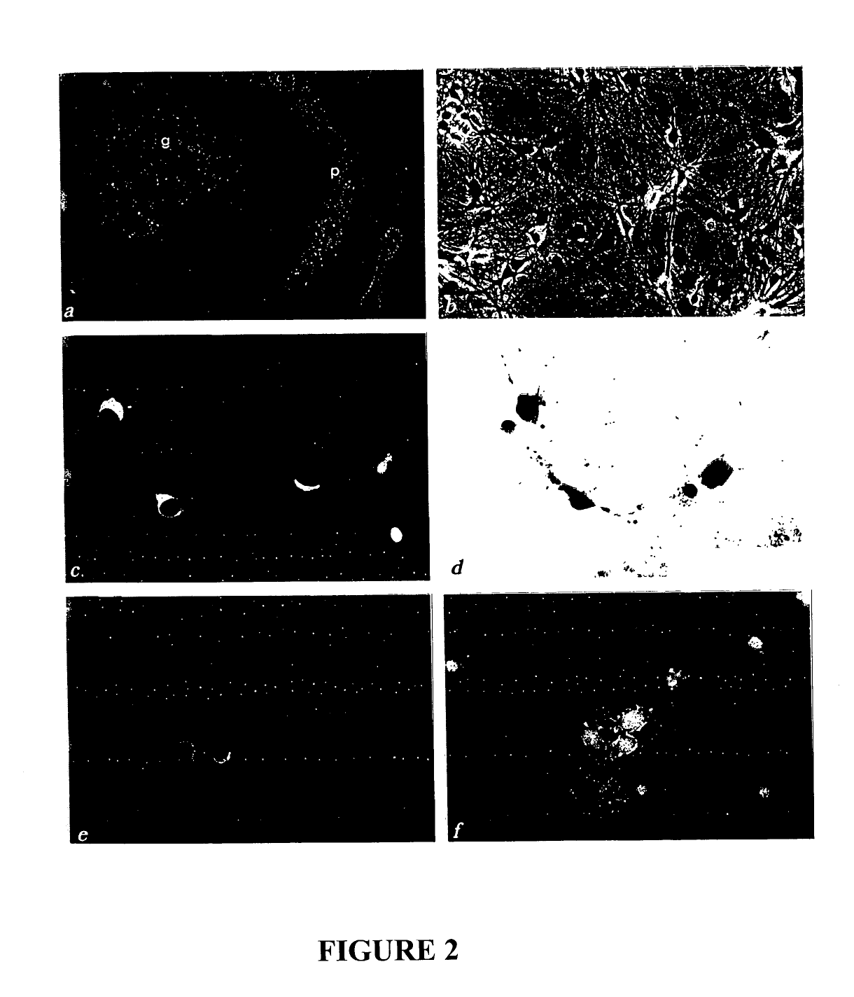

FIG. 1 shows that the monoclonal antibody G6E3 identifies specific neurons in the hippocampus (specifically the dentate gyrus, but fields CA1-CA3 stained robustly too). FIG. 2 is a composite showing the immunohistochemical staining of one MAb that was representative of the subset of MAbs that successfully passed through screening steps one and two. The hippocampal staining to both the dentate gyrus and neuronal fields CA3 through CA1 was robust. There was no apparent staining to glia. Cerebellar pyramidal neurons also stained positive with these antibodies. Thus, a subset of monoclonals exemplified by G6E3 were identified that recognized epitopes expressed on the cell surfaces of living hippocampal neurons and showed regionally restricted immunohistochemical staining to adult rat brain.

### Example 2

### Modulation of Synaptic Activity by Monoclonal Antibodies

To identify specific antibodies that could modulate cognitive function such as learning and memory, the antibodies described in Example 1 were subjected to further screening. The remaining panel of MAbs were screened using rat hippocampal slices and monitoring for the formation of long-term potentiation (LTP) followed by screening specifically for N-methyl-D-aspartate receptor modulation. LTP is a long-lasting enhancement of synaptic efficacy that is induced in the hippocampus by brief, high-frequency stimulation of the excitatory synapses (Bliss T. V. P. and Gardner-Medwin A. R. (1973) J. Physiol. 232, 357-374). The induction of LTP requires the activation of NMDA receptors by synaptically released glutamate (Harris E. W., Ganong A. H., and Cotman C. W. (1984) Brain Res. 323, 132-137) and coincident depolarization of the postsynaptic membranes (Kelso S. R., Ganong A. H., and Brown T. H. (1986) Proc. Natl. Acad. Sci USA 83, 5326-5330). The following studies focus on one monoclonal antibody, B6B21.

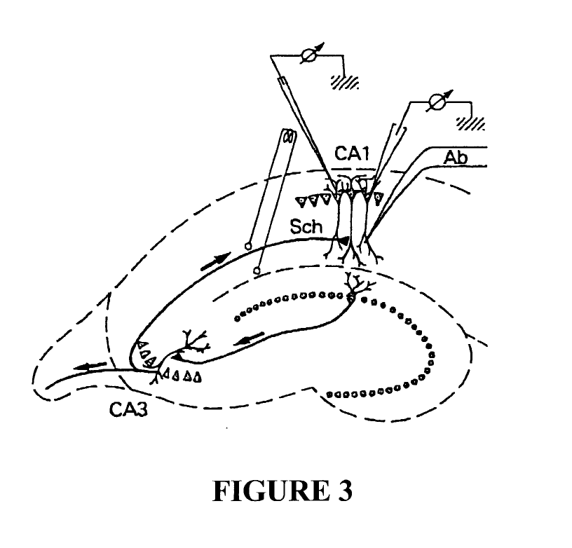

To test the hypothesis that B6B21 can interact with NMDA receptor-mediated plasticity, the effects of B6B21 treatment on the induction of LTP in CA1 pyramidal neurons in the hippocampus were tested. Recordings were taken extracellularly from two separate sites in the CA1 pyramidal cell body layer, with one of the two sites receiving pressure ejection of B6B21 to the apical dendritic field 15 min before high-frequency stimulation and the other serving as the paired control site. The experimental paradigm is illustrated in FIG. 3.

Tissue Preparation. Crude synaptic membranes were prepared from rat Hippocampi or from rat forebrains (male Sprague-Dawley rats) and washed extensively to remove endogenous amino acids, as previously described by Ransom and Stec ((1988) Brain Res. 444, 25-32). Briefly, the crude synaptic membranes were resuspended in 20 volumes of 5 mM Tris-HCl buffer pH 7.4 (for use in [³H]TCP-binding experiments), or in 20 volumes of 5 mM Tris-acetate buffer, pH 7.4 (for use in [³H]glycine-binding studies), and homogenized using a Polytron (Virtis shear; Virtis, N.Y., U.S.A.). Membranes were then pelleted by centrifugation at 48,000 g for 20 min. This step was repeated twice and the homogenate was stored at −70′ in the same buffer. Before each use, homogenates were thawed at room temperature, pelleted, and washed four additional times. For the [³H]glycine experiment, the pellet was first incubated for 30 min at 25° C. in 5 mM Tris-acetate buffer containing 0.04% Triton X-100 and then washed four times by homogenization and centrifugation. The final washed membranes were resuspended at concentrations of 2-3 mg/ml in either 5 mM Tris-HCl buffer or 5 mM Tris-acetate buffer.

TCP binding assay. Measurements of specific [³H]TCP binding were performed as described previously (Haring R., Kloog Y., Harshak-Felixbrodt N., and Sokolovsky M. (1987) Biochem. Biophys. Res. Commun. 142, 501-510). Final reaction of mixtures consisted of 50-100 μg of membrane protein in 200 μl of 5 mM Tris-HCl buffer and contained either [³H]TCP, or [³H]TCP and the appropriate concentration of NMDA-receptor ligands or MAbs. Reactions were initiated by the addition of the membranes to the reaction mixtures. Unless otherwise indicated, binding assays were performed under nonequilibrium conditions at 25° C. for 1 hr. Nonspecific binding was determined in parallel samples containing 100 μM unlabeled PCP. Binding reactions were terminated by filtration on Whatman GF/B glass filters that had been pretreated with 0.1% polyethyleneimine for 1 hr. The dissociation of [³H]TCP from its membrane-binding site was measured after equilibrating the receptors with 20 nM [³H]TCP for 120 min. The dissociation reaction was initiated by the addition of 100 μM unlabeled PCP in the presence and absence of NMDA-receptor ligands or MAb. Reactions were terminated immediately (zero time) and after incubation for the additional periods of time indicated.

Kinetic Analysis of Binding Data. Kinetic binding data were transformed into the form of first-order plots for the association kinetics in the following general equation:

(RL)_{eq}−(RL)/(RL)_{eq}=e^{(-k}_{obs}^{t)}

where k_{obs} is kₒₙL+k_{off}, RL is specifically bound [³H]TCP at time t, and RL_{eq} is specifically bound [³H]TCP at equilibrium. The logarithmic transformation of the kinetic data used to determine the association rate constant yielded biphasic plots in agreement with the previous reports (Bonhaus D. W. and McNamara J. O. (1988) Mol. PharmacoL 34, 250-255). The slopes of the initial phase of these curves (k_{obs}) were determined by linear regression analysis and plotted against [³H]TCP concentration. The dissociation rate constant was determined by linear regression analysis for the initial 30-40 min of the dissociation reaction, after plotting ln (Bₜ/Bₒ) versus time, where Bₒ corresponds to the amount of [³H]TCP bound at the termination of association and Bₜ represents the amount bound at time t.

Glycine binding assay. Measurements of specific [³H]glycine binding were performed essentially as described previously by Ransom and Stec (Ransom R. W. and Stec N. L. (1988) Brain Res. 444, 25-32). Briefly, membranes (100-150 μg of protein) were incubated at 25° C. for 30 min, in 50 mM Tris-acetate buffer containing the indicated concentrations of [³H]glycine in a final volume of 200 μl. Nonspecific binding was determined by including 1 mM glycine in the assay tubes. Binding was terminated by centrifugation (4° C.) at 12,000 g for 5 min. The pellets were rinsed with 3×1 ml of ice-cold buffer, the tip of the centrifuge tube was cut and placed in a 5-ml scintillation vial filled with Aquasol-2, and radioactivity was measured by liquid scintillation spectrometry. Data from ligand-binding experiments were fit to Scatchard plots where appropriate by using a least squares linear regression analysis. Binding studies represent the mean [±standard error of the mean (SEM)] of three to six experiments performed in triplicate. The data used to calculate the average varied by less than 10% from the mean. Statistical comparisons were performed using an unpaired, two-tailed Student's test.

MAb Preparation and Purification. mAbs were made by conventional techniques using BALB/c mice and NS-1 myeloma cells as previously described (Kohler G. and Milstein C. (1976) Immunol. 6, 511-519). Freshly dissected denate gyri of 5-day postnatal rats dentate served as immunogen (Moskal J. R. and Schaffner A. E. (1986) J. Neurosci. 6, 2045-2053). mAbs, derived from hybridoma culture supernatants, were purified by protein A-Sepharose affinity chromatography. Briefly, the pH of approximately 350 ml of culture supernatant was raised to 8.5 using 1 M Tris-HCl, pH 8.5, and loaded on a 5-ml Protein A Sepharose Cl-4B column pre-equilibrated with 50 mM Tris-HCl, pH 8.5. IgG was eluted from the column with 50 mM sodium-acetate buffer, pH 4.0, containing 1 M NaCl. The eluate was immediately neutralized with solid sodium bicarbonate, dialyzed overnight against 10 mM HEPES, pH 7.4, and concentrated using a Centriprep 30 (Amicon) concentrator. Concentrated antibody (typically 1-2 ml) was again dialyzed against two changes (2 L each) of HEPES buffer (as above) to be sure that there was no glycine contamination. To further ensure against contamination by amino acids or breakdown products of the antibody, a commercial preparation of murine IgG was treated identically and used as a control in all experiments. Thus, each antibody preparation was subjected to dialysis that would reduce the concentration of dialyzable contaminants, such as amino acids, by a factor of 4×10⁹. All buffers used were of the highest purity obtainable and no glycine, glutamate, or other NMDA receptor-relevant compound was used in any of the purification steps. Any possible artifact introduced by the purification, dialysis, concentration, and storage procedures was controlled for by the use of a control antibody subjected to identical conditions. Protein concentrations were determined at 280 nm using a molar extinction coefficient of 13 for a 1% IgG solution.

Slice Electrophysiology. Preparation of hippocampal slices and electrophysiological recording were performed as described previously (Stanton P. K., Sarvey J. M., and Moskal J. R. (1987) Proc. Natl. Acad. Sci USA 84, 1684-1688). Two extracellular recording sites in the CA1 pyramidal cell body layer (stratum pyramidale; 200-500 μm apart) were used in each slice, with one of the two sites selected for mAb application. The other site served as the control-evoked response. A bipolar glass-insulated platinum stimulating electrode (50-μm tip diameter) was placed in the Schaffer collateral/commissural axons in stratum radiatum on the CA3 side of both recording sites. Affinity-purified MAb was applied by pressure ejection (Picospritzer; General Valve, Fairfield, N.J., U.S.A.) from a pipette placed in the apical dendritic field (stratum radiatum) perpendicular to one of the somatic recording sites.

Antibody pipette ejection pressures and times were selected to supply 100-200 μm diameter droplets in air (12 psi/1 s; 1 psi=6.895 kPa). Two to four ejection were performed, spaced 1 min apart. Under these conditions, 0.1-1.0 μg of mAb (1 pmol mAb assuming Mᵣ=150,000 as the average molecular weight of an IgG 1 molecule) was delivered per ejection. No long-term alterations in low-frequency synaptic transmission, as measured either by population spike amplitude, initial epsp slope, or dendritic epsp recording, were observed after B6B21 application. Trains of high-frequency bursting stimuli were applied 15 min interval between antibody application and stimulation was used to ensure maximal binding of the antibody. Stimuli consisted of four trains of 100 Hz bursts given 15 s apart. Each burst had five stimuli and the interburst interval was 200 msec. Each train lasted 2 s for a total of 50 stimuli [5×10 bursts; see Stanton & Sejnowski (1989)]. The stimulus intensity used for high-frequency stimulation was selected to elicit ˜20-30% of maximal population spike amplitude, to yield clearly submaximal LTP in the absence of mAb application. Low-frequency test stimuli were applied every 5 s at various time points before and after tetanization. Extracellular responses were sampled by a digtal oscilloscope (Tektronix 2230) and stored on computer as the average of four responses. Spike amplitude was defined as the average of the amplitude from the peak early positivity to the peak negativity, and the amplitude from the peak negativity to the peak late positivity (Alger B. E. and Teyler T. J. (1976) Brain Res. 110, 463-480). LTP was defined as an amplitude increase of greater than 2 SD over prestimulated baselines measured 30 min after high-frequency stimulation.

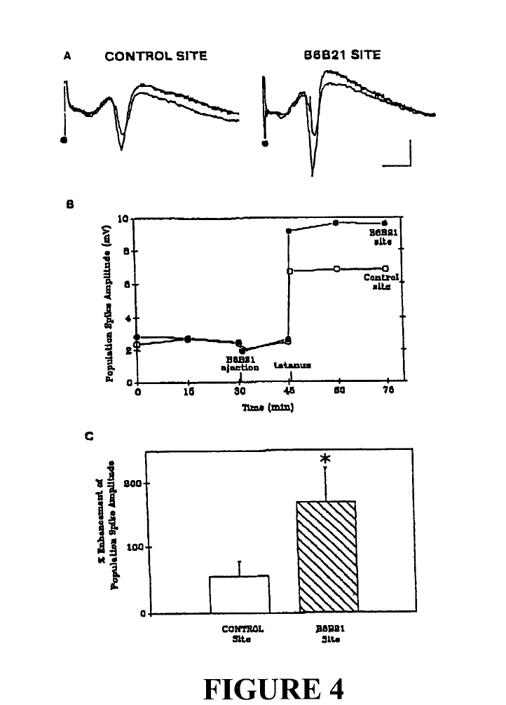

Stimulation of LTP by B6B21. FIG. 4 illustrates the enhancement of LTP induction produced by pretreatment with mAb B6B21 in the apical dendritic field in stratum radiatum (400-500 μm from the cell body layer). FIG. 4A shows LTP produced by a submaximal high-frequency Schaffer collateral stimulation at the control and B6B21-treated sites of a representative slice. The evoked population spike in the somatic layer is shown before (light traces) and 30 min after (dark traces) stimulation. In this slice, pretreatment with B6B21 doubled the amount of LTP elicited, compared with control site LTP evoked by the same stimuli. Comparison of the input/output (I/O) relations at the control and mAb-treated sites showed that B6B21 significantly enhanced the amount of LTP elicited by a submaximal tetanus irrespective of stimulus intensity within the linear portion of the I/O curve.

FIG. 4B shows the time course of a similar experiment, where B6B21 increased by 40% the LTP induced relative to the control, untreated site. At the start of the experiment, a Schaffer collateral stimulus intensity was selected that evoked almost identical amplitude population spikes at each of the two recording sites. Then, B6B21 was applied to the apical dendrites at one site selected at random (filled circles; 4×1-s application). Fifteen min later, four high-frequency stimulus trains (tetanus) were applied, which activated both the synapses treated with B6B21 and those at the untreated, control site (open circles). A rapid potentiation of evoked responses at both sites was observed, but the B6B21 site showed significantly more enhancement of spike amplitude relative to control. These experiments are summarized in FIG. 4C, showing the average percent enhancement of population spike amplitude (mean±SEM) elicited at control (solid bar) and B6B21-treated (hatched bar) recording sites in the CA1 pyramidal cell layer. In eight slices tested, B6B21 consistently and significantly enhanced LTP induction relative to untreated, control sites. (*p<0.05, paired t test). In these experiments, sites were selected at random for B6B21 application in each slice, so that in one-half of the experiments, the mAB site was more distal to the stimulating electrode and, in the other one-half, more proximal.

### Example 3

### Modulation of NMDA Receptor Function

A pharmacological approach was used to determine whether enhancement of LTP by B6B21 resulted from the direct interaction at the NMDA receptor-ion channel complex. The effect of B6B21 on specific [³H]TCP binding to washed rat hippocampal membranes was measured because agonists binding to glutamate and glycine site associated with the NMDA receptor alters binding interactions at the PCP site.

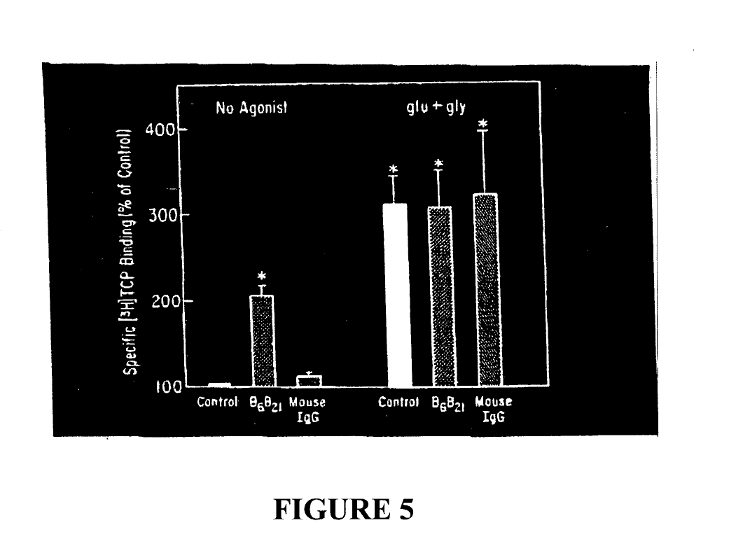

As shown in FIG. 5, affinity-purified B6B21 (25 μg/ml) elevated specific [³H]TCP binding to NMDA receptors twofold compared with control binding (*p<0.05, Student's t test; n=6). By comparison, a commercial preparation of affinity-purified mouse IgG did not significantly elevate [³H]TCP binding when compared with control binding. As described in Example 2, affinity-purified B6B21 and commercial preparation of murine IgG were exhaustively dialyzed to eliminate any compounds (such as glycine) that could potentially interfere with the binding or give false-positive results. To further control for any artifacts due to nonspecific antibody effects, a commercial preparation of murine IgG was treated identically to B6B21 preparation and used as a control. Each batch of B6B21 was prepared in parallel with the batch of commercial IgG using identical conditions, buffers, and affinity columns. The combined addition of maximally stimulating concentrations (50 μM) of glutamate, glycine, and magnesium caused a threefold increase in [³H]TCP binding (*p<0.05, Student's t test; n=7). As shown in FIG. 5, no further increase in binding was observed after the addition of B6B21 or mouse IgG to the mixture of glutamate, glycine, and magnesium, suggesting that B6B21 specifically interacts at the NMDA receptor-ion channel complex.

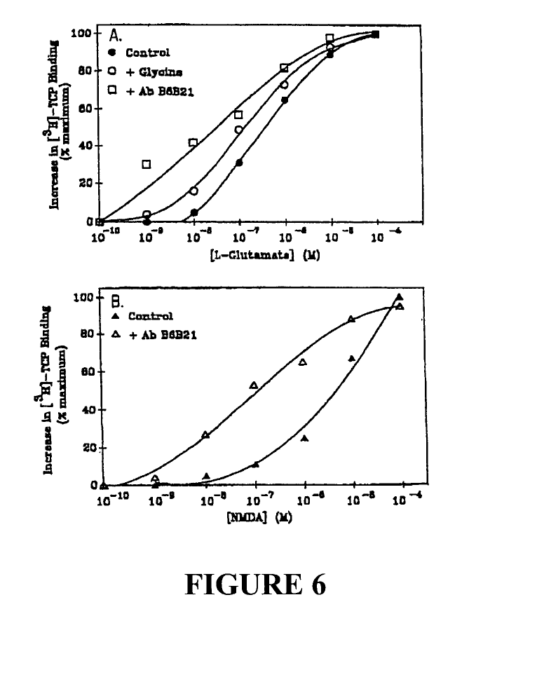

Further measurements of specific [³H]TCP binding were made to discriminate between a B6B21 interaction at NMDA receptor-associated glutamate and/or glycine sites. A dose dependent stimulatory effect of glutamate on [³H]TCP binding is shown in FIG. 6A. Glutamate maximally stimulated specific [³H]TCP binding at concentrations in excess of 10 μM. The concentration of glutamate required to stimulate 50% of maximal binding (EC₅₀) was 0.7 μM. Co-addition of either glycine or B6B21 potentiated the glutamate effect additively (at concentrations that give maximal effects for these compounds), suggesting that the effects of glycine or B6B21 were mediated at a binding site distinct from that of glutamate.

In previous reports, measurements of [³H]TCP binding at equilibrium has not yielded a change in the maximal number of PCP-binding sites (Bₘₐₓ) after the addition of activating ligands (Reynolds I. J., Murphy S. N., and Miller R. J. (1987) Proc. Natl. Acad. Sci. USA 84, 7744-7748). In the present study, because assay conditions were not at equilibrium (1 h, 25° C.), specific [³H]TCP binding was stimulated from 63 to 132 fmol/mg of protein by the addition of a maximally stimulating concentration of glutamate, as shown in FIG. 6. Both glycine and B6B21 augmented this effect, increasing [³H]TCP binding to 310 and 286 fmol/mg of protein, respectively. The ability of either glycine (50 μM) or B6B21 (25 μM, assuming Mᵣ=150,000) to increase specific [³H]TCP binding in the absence of added glutamate (155 and 138 fmol/mg of protein) suggests that the membrane preparations were incompletely washed. EC₅₀ values for glutamate stimulation in these experiments were reduced in the presence of 50 μM glycine, from 0.7 to 0.13 μM, or by 25 μM of B6B21 to 0.038 μM (FIG. 4A). Similar results were obtained when NMDA was substituted for glutamate, as seen in FIG. 6B. The EC₅₀ value for NMDA was reduced 57-fold in the presence of 25 μg/ml of B6B21, as seen in FIG. 6B. By comparison, the ability of glycine to stimulate specific [³H]TCP binding was unchanged after the addition of B6B21 to membranes.

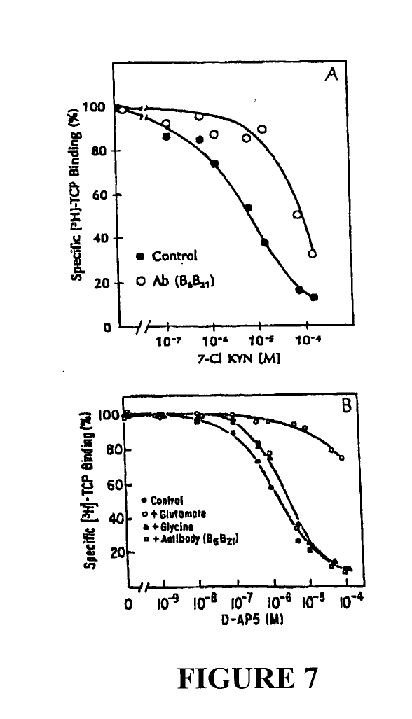

Further experiments were performed with 7-Cl KYNA, which has been reported to attenuate NMDA-receptor function mediated at the strychnine-insensitive glycine site (Kemp J. A., Foster A. C., Leeson P. D., Priestley R., Tridgett R., Iversen L. L., and Woodruff G. N. (1988) Proc. Natl. Acad. Sci USA 85, 6547-6550). As shown in FIG. 7A, 7-Cl KYNA dose-dependently inhibited [³H]TCP binding. The IC₅₀ value obtained from these experiments was 6 μM, in a good agreement with previously reported values (Kloog Y., Lamdani-Itkin H., and Sokolovsky M. (1990) J. Neurochem. 54, 1576-1583). In the presence of B6B21 (40 μg/ml), the IC₅₀ for 7-CL KYNA was shifted to 60 μM. These results demonstrate that B6B21 selectively mimics the effects of glycine, but not those of glutamate or NMDA. In further support of this view, we found that the ability of the NMDA antagonist D-AP5 to inhibit specific [³H]TCP binding is strongly reduced by glutamate, but not either glycine or B6B21 (FIG. 7B). In these experiments, the IC₅₀ value for D-AP5 inhibition was increased from 1.4 μM in the absence of glutamate to greater than 100 μM in the presence of 50 μM glutamate. Neither glycine nor B6B21 could abolish the inhibitory effect of D-AP5.

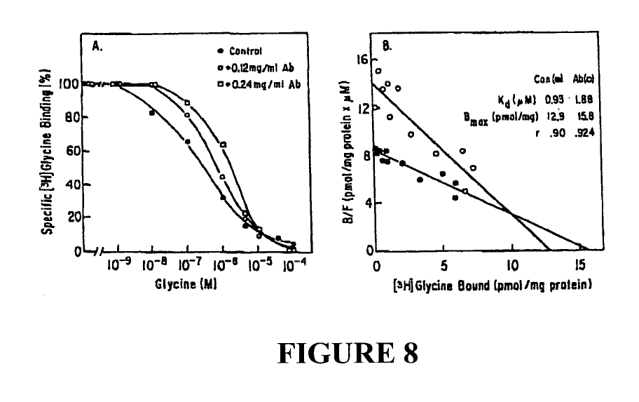

Inhibition of Glycine Binding by mAb B6B21. To further test the hypothesis that B6B21 interacts with the glycine-binding site of the NMDA-receptor complex, we directly assayed the ability of B6B21 to alter glycine binding. Labeling of the glycine recognition site with [³H]glycine was saturable and reversible (FIG. 8). Radioligand binding was displaced by glycine (IC₅₀=0.4 μM) and by D-serine (IC₅₀=1.2 μM). Scatchard analysis of the direct radioligand-binding curves (FIG. 8B) demonstrated the existence of a single population of sites. Values of K_{D} (0.93 μM) and Bₘₐₓ (12.9 pmol/mg of protein) were similar to those measured previously in rat forebrain membrane preparation (Lester et al., 1989). The addition of B6B21 caused a dose-dependent shift of the inhibition curve to higher concentration. IC₅₀ values obtained in the presence of 0.12 or 0.24 mg/ml of B6B21 were increased to 1.1 and 3 μM, respectively (FIG. 8A). By comparison, the addition of mouse IgG (0.24 mg/ml) did not alter the displacement of [³H]glycine binding (IC₅₀=0.55 μM). B6B21 (0.24 mg/ml) increased the apparent K_{D} for [³H]glycine at this site by 188% (the K_{D} increased to 1.9 μM) with no significant change in the Bₘₐₓ (FIG. 8B). Preliminary observation also demonstrated a direct competition of B6B21 with [³H]glycine binding (the appropriate ICₘₐₓ value was 0.24 mg/ml; 1.6 μM). These data directly demonstrated a B6B21 interaction with the strychnine-insensitive binding site on the NMDA-receptor complex.

The enhancement of LTP together with the demonstration that B6B21 modulates the NMDA receptor at the glycine site strongly suggested that this antibody could have therapeutic potential.

### Example 4

### B6B21 Effects on Learning and Memory

To examine the in vivo actions of B6B21 on learning and memory, a hippocampus-dependent learning task was performed with rabbits. The hippocampus is particularly rich in NMDA receptors. Long-term potentiation of Schaffer collateral or perforant-path synapses is blocked by competitive antagonists of the NMDA binding site or of its strychnine-insensitive glycine coagonist site. NMDA antagonists (both competitive and non-competitive) inhibit acquisition in behavioral learning tasks at doses that do not impair performance. Trace conditioning requires the formation of a short-term “memory trace” of the conditions stimulus to bridge the interstimulus interval between conditioned and unconditioned stimulus to successfully form an association. The hippocampus can serve this and other mnemonic functions. Acquisition of the 500 ms trace eye-blink conditioning task used here is blocked by hippocampal lesions in rabbits. When B6B21 was injected into the lateral ventricles, this large glycoprotein (Mᵣ=150 kD) was unlikely to penetrate distal structures within a reasonable time for action. But the ventricular system gave relatively direct access to the hippocampus. Though hippocampal function is not an absolute requirement for all forms of learning, enhancement of hippocampal function can be of benefit even in tasks that are not dependant on the hippocampus.

Lateral ventricular guide cannulae were surgically implanted bilaterally in rabbits, which were then fitted with restraining head bolts. After recovery and environment habituation, rabbits were trained in pairs in separate, darkened and sound-attenuated chambers. Training continued until a certain criterion level of 80% of paired stimulus presentations resulted in the conditioned response (80% CR).

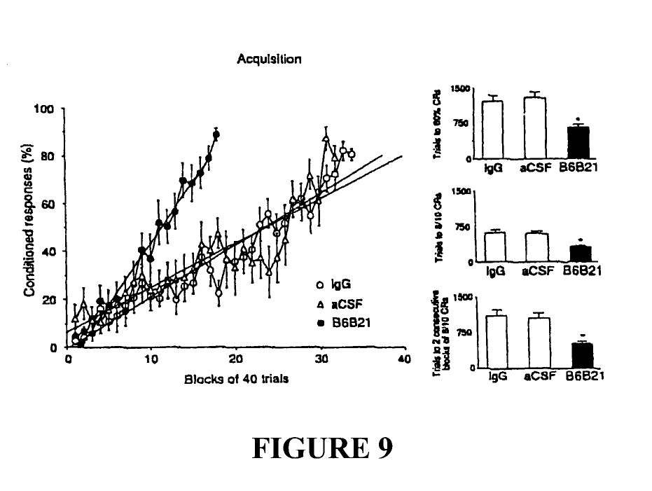

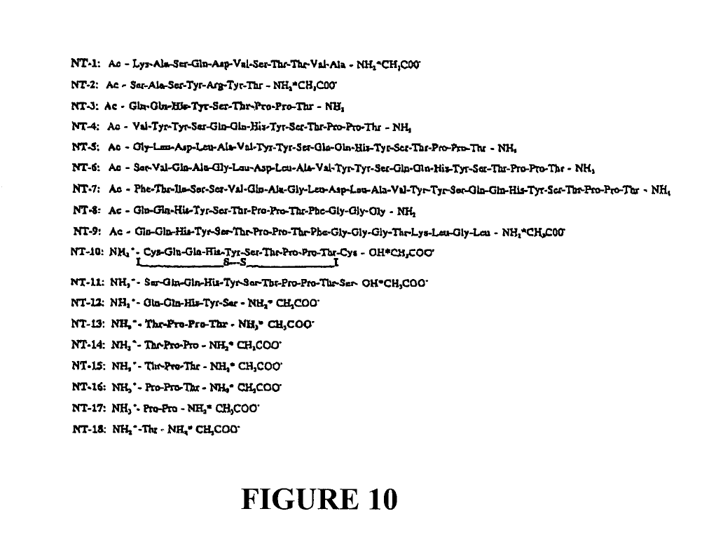

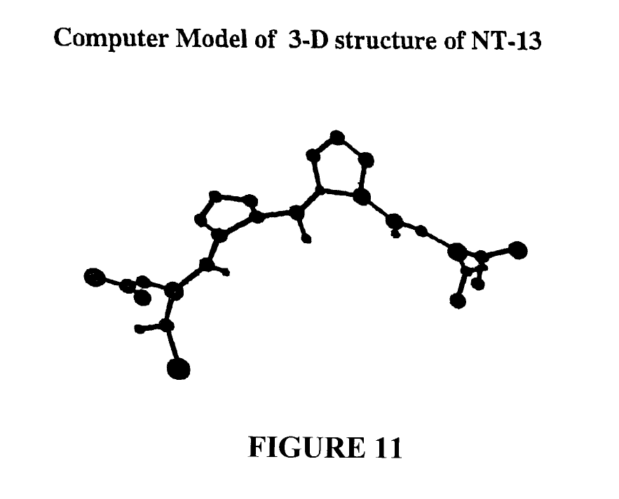

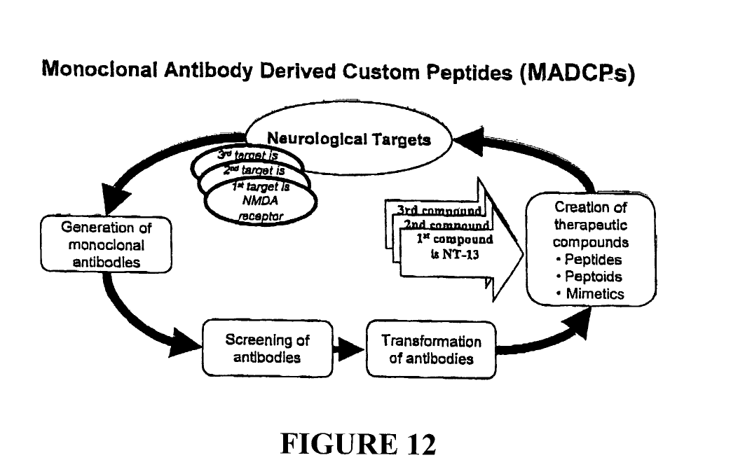

It can be seen in FIG. 9 that intraventricular infusion of B6B21 greatly accelerated acquisition of trace conditioning as compared with ether IgG or artificial cerebrospinal fluid control treatments (F₂=15.14, P<0.0003). In every case, B6B21-treated animals learned much more rapidly than their paired controls. No effects of B6B21 treatment on the unconditioned response amplitude during conditioning were observed (P<0.68).

Clearly then, B6B21 was able to markedly enhance learning without any apparent side effects such as seizures, psychotic episodes, or related central nervous system functional toxicities. However, it was also clear that B6B21 was too large a molecule—a multisubunit glycoprotein with a Mᵣ=150,000 kD—to be useful therapeutically.

### Example 5

### Cloning of the Complimentarity Determining Regions (CDRs) of Monoclonal Antibody B6B21

The amino acids that comprise the CDRs of an antibody molecule determine its binding specificity. Synthetic peptides, derived from these amino acid sequences have been demonstrated to possess biological activity similar to that of the intact antibody (Novotny et al., 1986; Kang et al., 1988; Williams et al., 1988, 1989, 1991; Saragovi et al., 1991; Welling et al., 1991). To design B6B21 antibody mimetics, we have cloned the hypervariable regions of the light chain of B6B21 using reverse transcriptase-polymerase chain reaction (RT-PCR) technology (Yamamoto and Gurney, 1990).

FIG. 12 illustrates monoclonal antibody-derived custom peptides (MADCP). This cartoon depicts the general method that can be used to generate therapeutically useful compounds via monoclonal antibodies. By identifying appropriate targets as immunogens coupled with the proper screening methods, antibodies can be generated and the hypervariable regions (CDRs) can be cloned and sequenced. From this information, small peptides can easily be synthesized that posses therapeutic value as well. Moreover, thousands of variants can be readily synthesized via amino acid substitutions and because these peptides tend to be quite small, non-peptide peptidomimetics can also be synthesized using methods known in the art as described above.

B6B21 cDNA was synthesized using poly A+ RNA isolated from B6B21 hybridoma cells using a modification of previously published methods (Yamamoto and Gurney, 1990). PCR was performed using 6 different sets of degenerate immunogloblin heavy and light chain primers (mouse Ig-primers, Novagen, Madison Wis.). Cloning and sequencing of these reaction products showed that they both were derived from IgG transcripts found in NS-1 cells, and thus were not specific to B6B21 hybridoma cells.

Peptide Synthesis. After cloning and sequencing the light chain variable region of B6B21, three hypervariable region peptides were identified and synthesized (FIG. 10); peptides NT-1, NT-2, and NT-3. NT-3 was found to significantly enhance [³H]MK-801 binding (see FIG. 4 for the description of the modified NMDA receptor-specific, glycine site-dependent, [³H]MK-801 binding assay) comparable to both B6B21 and the positive control, D-cycloserine. NT-3, at 10⁻⁸-10⁻⁷, stimulate [³H]MK-801 binding approximately 120% above control. B6B21 gave approximately a 150% increase above control at 0.1 μg/ml (0.67 nM given an Mᵣ=150,000 for B6B21), whereas D-cycloserine was optimally active (approx. 130% above control) at 10⁻⁵ M.

Based on these results, a series of NT-3 variants were synthesized and tested. NTs-4, 5, 6 and 7 contain additional amino acids at the N-terminal of NT-3 and NTs 8 and 9 contain additional amino acids at the C-terminal of NT-3. None of these modifications led to increased [³H]MK-801 binding and, in fact, were often much less efficacious than NT-3. NT-10 is the cyclized version of NT-3, and NT-11 is its control. NT-10 and NT-11 both showed activity comparable to NT-3, but no pronounced increase in efficacy was observed. NT-12 and NT-13 are the penta- and tetrapeptides, respectively, created by cleavage between the tyrosine and serine residues of NT-3. NT-12 did not enhance [³H]MK-801 binding. NT-13, however, stimulated [³H]MK-801 binding to 130% of control at 10⁻⁶ M. NT-13 also was stable to repeated freezing and thawing.

FIG. 11 shows NT-13, proline-threonine-threonine-proline, as it likely appears in solution. It should be noted that the molecule exists in a betal-type turn that is consistent with the literature that protein-protein interactions often take place through peptide sequences with beta 1-type turns.

The NT-13 peptide has been shown to cross the blood brain barrier and can be delivered either intravenously or trans-nasally. NT-13 appears to be relatively non-toxic in that up to 500 mg/Kg of peptide delivered IV has had no obvious side effects. Finally, recent studies have shown that when rats were given IV doses of NT-13 up to 2 mg/kg, a statistically significant increase in learning occurred both in the trace eyeblink paradigm used with the mAb, B6B21 and in the Morris Water Maze (Gamelli et al., Soc. Neuroscience, 2001). Moreover, recent studies using a gerbil model of hypoxia have shown that 5 mg/kg of NT-13, given before hypoxic insult or up to five hours after, was able to completely protect the hippocampus from neural degeneration associated with stroke.

What is claimed is:

1. A method for identifying a phenotypically active binding agent, comprising:
(a) contacting a first cell with a first binding agent, wherein said first cell comprises a binding target;
(b) detecting binding of said first binding agent to said first cell;
(c) preparing a second binding agent, wherein said second binding agent is derived from said first binding agent;
(d) contacting a second cell with said second binding agent; wherein said second cell comprises said binding target;
(e) detecting a phenotypic change in said second cell in response to said contact with said second binding agent; wherein said phenotypic change in said second cell is in response to said contact with said second binding agent;
whereby said second binding agent is identified as a phenotypically active binding agent.

2. The method of claim 1, wherein said first cell is a neural cell.

3. The method of claim 2, wherein said second cell is a neural cell.

4. The method of claim 2, wherein said first binding agent is a protein or a peptide.

5. The method of claim 4, wherein the second binding agent comprises a peptide, wherein said peptide comprises a sequence of contiguous amino acids that is substantially identical to a region of contiguous amino acids in the first binding agent.

6. The method of claim 5, wherein said peptide comprises a sequence of at least 3 contiguous amino acids identical to a region of contiguous amino acids in said first binding agent.

7. The method of claim 5, wherein said peptide comprises a sequence of at least 4 contiguous amino acids identical to a region of contiguous amino acids in said first binding agent.

8. The method of claim 5, wherein said peptide comprises a sequence of at least 5 contiguous amino acids identical to a region of contiguous amino acids in said first binding agent.

9. The method of claim 5, wherein said peptide comprises a sequence of at least 9 contiguous amino acids identical to a region of contiguous amino acids in said first binding agent.

10. The method of claim 2, wherein said first binding agent is an antibody molecule or fragment thereof.

11. The method of claim 2, wherein said first binding agent is a monoclonal antibody or fragment thereof.

12. The method of claim 11, wherein said second binding agent comprises the complementarity determining region of said first binding agent, or a fragment thereof.

13. The method of claim 2, wherein the first binding agent is a fragment of an antibody expressed on a phage particle.

14. The method of claim 2, wherein the detection of binding of said first binding agent of (b) occurs by a method comprising detecting a phenotypic change in said first cell in response to contact with said binding agent.

15. The method of claim 2, wherein said second binding agent is a peptidomimetic.

16. The method of claim 2, wherein said second binding agent comprises a sequence of contiguous amino acids that is substantially identical to a retro-inverso peptide corresponding to a region of contiguous amino acids in said first binding agent.

17. The method of claim 2, wherein said second binding agent is prepared by a method comprising isolating a nucleic acid encoding the first binding agent.

18. The method of claim 17, wherein the first binding agent is expressed by a hybridoma cell.

19. The method of claim 18, wherein said nucleic acid is a fragment of genomic DNA isolated from said hybridoma cell.

20. The method of claim 18, wherein said nucleic acid is a fragment of cDNA prepared from the mRNA of said hybridoma cell.

21. The method of claim 2, wherein said binding target is a cell-surface receptor.

22. The method of claim 21, wherein said cell-surface receptor is an ion channel gate.

23. The method of claim 21, wherein said cell-surface receptor is the N-methly-D-aspartate receptor.

24. The method of claim 2, wherein said phenotypic change in second cell comprises induction of long-term potentiation.

25. The method of claim 2, wherein the second binding agent is an organic molecule.

26. A method for identifying a phenotypically active binding agent, comprising:
(a) identifying a binding target;
(b) contacting a first cell with a first binding agent, wherein said first cell comprises said binding target;
(c) detecting binding of said first binding agent to said first cell;
(d) preparing a second binding agent, wherein said second binding agent is derived from said first binding agent;
(e) contacting a second cell with said second binding agent; wherein said second cell comprises said binding target;
(f) detecting a phenotypic change in said second cell in response to said contact with said second binding agent; wherein said phenotypic change in said second cell is in response to said contact with said second binding agent;
whereby said second binding agent is identified as a phenotypically active binding agent.

27. A method for identifying a phenotypically active binding agent, comprising:
(a) identifying a first binding agent, wherein first binding agent binds to a binding target,
(b) preparing a second binding agent, wherein said second binding agent is derived from said first binding agent;
(c) contacting a cell with said second binding agent; wherein said cell comprises said binding target;
(d) detecting a phenotypic change in said cell in response to said contact with said second binding agent; wherein said phenotypic change in said second cell is in response to said contact with said second binding agent;
whereby said second binding agent is identified as a phenotypically active binding agent.

28. The method of claim 27, wherein said cell is a neural cell.

29. The method of claim 28, wherein said first binding agent is a protein or a peptide.

30. The method of claim 28, wherein said second binding agent comprises a peptide, wherein said peptide comprises a sequence of contiguous amino acids that is substantially identical to a region of contiguous amino acids in said first binding agent.

31. The method of claim 28, wherein said first binding agent is a monoclonal antibody.

32. The method of claim 28, wherein said second binding agent is a peptidomimetic.

33. The method of claim 28, wherein said binding target is a cell-surface receptor.

34. The method of claim 33, wherein said cell-surface receptor is the N-methyl-D-aspartate receptor.

35. The method of claim 28, wherein said phenotypic change in said second cell comprises induction of long-term potentiation.

36. A method for identifying a phenotypically active binding agent, comprising:
(a) contacting a first cell with a first binding agent, wherein said first cell comprises a binding target and wherein said first binding agent is encoded by a nucleic acid;
(b) detecting binding of said first binding agent to said first cell;
(c) preparing a nucleic acid library, wherein said nucleic acid library comprises said nucleic acid encoding said first binding agent, or a fragment thereof;
(d) preparing a second binding agent, wherein said second binding agent is prepared by utilizing said nucleic acid library;
(e) contacting a second cell with said second binding agent; wherein said second cell comprises said binding target;
(f) detecting a phenotypic change in said second cell in response to said contact with said second binding agent; wherein said phenotypic change in said second cell is in response to said contact with said second binding agent;
whereby said second binding agent is identified as a phenotypically active binding agent.

37. The method of claim 36, wherein said first cell is a neural cell.

38. The method of claim 37, wherein said nucleic acid library is derived from a genomic library of a hybridoma cell that expresses a monoclonal antibody.

39. The method of claim 37, wherein said nucleic acid library is derived from a cDNA library of a hybridoma cell that expresses a monoclonal antibody.

40. The method of claim 37, wherein said nucleic acid library is a phage display library.

41. The method of claim 37, wherein said nucleic acid library comprises a stochastically generated library.

42. The method of claim 37, wherein said nucleic acid library is modified by gene shuffling.

43. The method of claim 37, wherein said second binding agent is prepared by gene shuffling said nucleic acid library.

44. The method of claim 37, wherein said second binding agent is a peptidomimetic.

45. A phenotypically active binding agent identified by the method of claim 2.

46. A phenotypically active binding agent identified by the method of claim 5.

47. A phenotypically active binding agent identified by the method of claim 12.

48. A phenotypically active binding agent identified by the method of claim 15.

49. A phenotypically active binding agent identified by the method of claim 18.

50. A phenotypically active binding agent identified by the method of claim 21.

51. A phenotypically active binding agent identified by the method of claim 23.

52. A phenotypically active binding agent identified by the method of claim 24.

53. A phenotypically active binding agent identified by the method of claim 26.

54. A phenotypically active binding agent identified by the method of claim 28.

55. A phenotypically active binding agent identified by the method of claim 30.

56. A phenotypically active binding agent identified by the method of claim 31.

57. A phenotypically active binding agent identified by the method of claim 32.

58. A phenotypically active binding agent identified by the method of claim 33.

59. A phenotypically active binding agent identified by the method of claim 34.

60. A phenotypically active binding agent identified by the method of claim 35.

61. A phenotypically active binding agent identified by the method of claim 37.

62. A phenotypically active binding agent identified by the method of claim 40.

63. A phenotypically active binding agent identified by the method of claim 44.
